(19) United States
(12) Patent Application Publication
(10) Pub. No.: US 2010/0317575 A1
(43) Pub. Date: Dec. 16, 2010

(54) PHARMACEUTICAL COMPOSITION COMPRISING A PYRAZOLE-O-GLUCOSIDE DERIVATIVE

(75) Inventors: Sabine Pinnetti, Biberach (DE); Ruediger Streicher, Biberach (DE); Leo Thomas, Biberach (DE); Klaus Dugi, Dresden (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignees: BOEHRINGER INGELHEIM INTERNATIONAL GMBH, Ingelheim (DE); AJINOMOTO CO., INC., Tokyo (JP)

(21) Appl. No.: 12/521,644

(22) PCT Filed: Jan. 18, 2008
(86) PCT No.: PCT/EP08/50533

§ 371 Date: Sep. 18, 2009

(30) Foreign Application Priority Data
Jan. 19, 2007 (EP) 07100779.3

(51) Int. Cl. A61K 31/7056; A61P 3/10; A61K 38/28; A61K 38/26; A61K 38/16; A61K 38/22; A61P 3/04; A61P 27/12; A61P 9/00; A61P 9/10

(52) U.S. Cl. 514/6.5; 514/27; 514/7.2; 514/6.8; 514/7.3

(57) ABSTRACT

The invention relates to a pharmaceutical composition comprising a pyrazole-O-glucoside derivative selected from the group of compounds (1) to (29) according to claim 1 in combination with at least one second therapeutic agent which is suitable in the treatment or prevention of one or more conditions selected from type 1 diabetes mellitus, type 2 diabetes mellitus, impaired glucose tolerance and hyperglycemia. In addition the present invention relates to methods for preventing or treating of metabolic disorders and related conditions.

### TECHNICAL FIELD OF THE INVENTION

The invention relates to a pharmaceutical composition comprising a pyrazole-O-glucoside derivative selected from the group of compounds (1) to (29) as defined hereinafter in combination with at least one second therapeutic agent which is suitable in the treatment or prevention of one or more conditions selected from type 1 diabetes mellitus, type 2 diabetes mellitus, impaired glucose tolerance, impaired fasting blood glucose and hyperglycemia.

Furthermore the invention relates to methods
for preventing, slowing progression of, delaying, or treating a metabolic disorder;
for improving glycemic control and/or for reducing of fasting plasma glucose, of postprandial plasma glucose and/or of glycosylated hemoglobin HbA1c;
for preventing, slowing, delaying or reversing progression from impaired glucose tolerance, impaired fasting blood glucose, insulin resistance and/or from metabolic syndrome to type 2 diabetes mellitus;
for preventing, slowing progression of, delaying or treating of a condition or disorder selected from the group consisting of complications of diabetes mellitus;
for reducing the weight or preventing an increase of the weight or facilitating a reduction of the weight;
for preventing or treating the degeneration of pancreatic beta cells and/or for improving and/or restoring the functionality of pancreatic beta cells and/or restoring the functionality of pancreatic insulin secretion;
for preventing, slowing, delaying or treating diseases or conditions attributed to an abnormal accumulation of liver fat;
maintaining and/or improving the insulin sensitivity and/or for treating or preventing hyperinsulinemia and/or insulin resistance,

in patients in need thereof characterized in that a pyrazole-O-glucoside derivative selected from the group of compounds (1) to (29) as defined hereinafter is administered in combination or alternation with at least one second therapeutic agent as defined hereinafter.

In addition the present invention relates to the use of a pyrazole-O-glucoside derivative selected from the group of compounds (1) to (29) as defined hereinafter for the manufacture of a medicament for use in a method as described hereinbefore and hereinafter.

In addition the present invention relates to the use of at least one second therapeutic agent as defined hereinafter for the manufacture of a medicament for use in a method as described hereinbefore and hereinafter.

The invention also relates to a use of a pharmaceutical composition according to this invention for the manufacture of a medicament for use in a method as described hereinbefore and hereinafter.

### BACKGROUND OF THE INVENTION

The European Patent application EP 1 338 603 A1 describes novel pyrazole-O-glycoside derivatives. The pyrazole-O-glycoside derivatives are proposed as inducers of urinary sugar excretion and as medicaments in the treatment of diabetes.

The European Patent application EP 1 500 403 A1 describes a combination of an inhibitor of renal glucose reabsorption and a hypoglycemic agent.

Renal filtration and reuptake of glucose contributes, among other mechanisms, to the steady state plasma glucose concentration and can therefore serve as an antidiabetic target. Reuptake of filtered glucose across epithelial cells of the kidney proceeds via sodium-dependent glucose cotransporters (SGLTs) located in the brush-border membranes in the proximal tubuli along the sodium gradient⁽¹⁾. There are at least 3 SGLT isoforms that differ in their expression pattern as well as in their physico-chemical properties⁽²⁾. SGLT2 is exclusively expressed in the kidney⁽³⁾, whereas SGLT1 is expressed additionally in other tissues like intestine, colon, skeletal and cardiac muscle^{(4;5)}. SGLT3 has been found to be a glucose sensor in interstitial cells of the intestine without any transport function⁽⁶⁾. Potentially, other related, but not yet characterized genes, may contribute further to renal glucose reuptake^{(7,8,9)}. Under normoglycemia, glucose is completely reabsorbed by SGLTs in the kidney, whereas the reuptake capacity of the kidney is saturated at glucose concentrations higher than 10 mM, resulting in glucosuria (“diabetes mellitus”). This threshold concentration can be decreased by SGLT2-inhibition. It has been shown in experiments with the SGLT inhibitor phlorizin that SGLT-inhibition will partially inhibit the reuptake of glucose from the glomerular filtrate into the blood leading to a decrease in blood glucose concentrations and to glucosuria^{(10;11)}. (1) Wright, E. M. (2001) Am. J. Renal Physiol. 280, F10-F18;(2) Wright, E. M. et al. (2004) Pflugers Arch. 447(5):510-8;(3) You, G. et al. (1995) J. Biol. Chem. 270 (49) 29365-29371;(4) Pajor A M, Wright E M (1992) J Biol. Chem. 267(6):3557-3560;(5) Zhou, L. et al. (2003) J. Cell. Biochem. 90:339-346;(6) Diez-Sampedro, A. et al. (2003) Proc. Natl. Acad. Sci. USA 100(20), 11753-11758;(7) Tabatabai, N. M. (2003) Kidney Int. 64, 1320-1330;(8) Curtis, R. A. J. (2003) US Patent Appl. 2003/0054453;(9) Bruss, M. and Bonisch, H. (2001) Cloning and functional characterization of a new human sugar transporter in kidney (Genbank Acc. No. AJ305237);(10) Rossetti, L. Et al. (987) J. Clin. Invest. 79, 1510-1515;(11) Gouvea, W. L. (1989) Kidney Int. 35(4):1041-1048.

Type 2 diabetes is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk.

After long duration of disease, most patients with type 2 diabetes will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c ˜0.9%). In addition, even in patients within the intensive treatment arm glycemic control deteriorated significantly over time and this was attributed to deterioration of β-cell function. Importantly, intensive treatment was not associated with a significant reduction in macrovascular complications, i.e. cardiovascular events.

Therefore there is an unmet medical need for methods, medicaments and pharmaceutical compositions with a good efficacy with regard to glycemic control, with regard to disease-modifying properties and with regard to reduction of cardiovascular morbidity and mortality while at the same time showing an improved safety profile.

### AIM OF THE PRESENT INVENTION

The aim of the present invention is to provide a pharmaceutical composition and method for preventing, slowing progression of, delaying or treating a metabolic disorder.

A further aim of the present invention is to provide a pharmaceutical composition and method for improving glycemic control in a patient in need thereof.

Another aim of the present invention is to provide a pharmaceutical composition and method for preventing, slowing or delaying progression from impaired glucose tolerance (IGT), impaired fasting blood glucose (IFG), insulin resistance and/or metabolic syndrome to type 2 diabetes mellitus.

Yet another aim of the present invention is to provide a pharmaceutical composition and method for preventing, slowing progression of, delaying or treating of a condition or disorder from the group consisting of complications of diabetes mellitus.

A further aim of the present invention is to provide a pharmaceutical composition and method for reducing the weight or preventing an increase of the weight in a patient in need thereof.

Another aim of the present invention is to provide a new pharmaceutical composition with a high efficacy for the treatment of metabolic disorders, in particular of diabetes mellitus, impaired glucose tolerance (IGT), impaired fasting blood glucose (IFG), and/or hyperglycemia, which has good to very good pharmacological and/or pharmacokinetic and/or physicochemical properties.

Further aims of the present invention become apparent to the one skilled in the art by description hereinbefore and in the following and by the examples.

### SUMMARY OF THE INVENTION

Within the scope of the present invention it has now surprisingly been found that a pharmaceutical composition comprising a pyrazole-O-glucoside derivative selected from the group of compounds (1) to (29), or prodrugs thereof, or pharmaceutically acceptable salts thereof, as defined hereinafter can advantageously be used in combination with at least one second therapeutic agent which is suitable in the treatment or prevention of one or more conditions selected from type 1 diabetes mellitus, type 2 diabetes mellitus, impaired glucose tolerance (IGT), impaired fasting blood glucose (IFG) and hyperglycemia for preventing, slowing progression of, delaying or treating a metabolic disorder, in particular in improving glycemic control in patients. This opens up new therapeutic possibilities in the treatment and prevention of type 2 diabetes mellitus, overweight, obesity, complications of diabetes mellitus and of neighboring disease states.

Therefore in a first aspect the present invention provides a pharmaceutical composition comprising a pyrazole-O-glucoside derivative selected from the group of compounds (1) to (29) consisting of
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(16) 4-(2-fluoro-4-methoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
(21) 4-(4-ethyl-benzyl)-1-isopropyl-5-trifluoromethyl-3-β-D-glucopyranos-1-yloxy-1H-pyrazole;
(22) 4-(4-bromo-benzyl)-1-isopropyl-5-trifluoromethyl-3-β-D-glucopyranos-1-yloxy-1H-pyrazole;
(23) 4-(4-ethyl-benzyl)-1-cyclobutyl-5-trifluoromethyl-3-β-D-glucopyranos-1-yloxy-1H-pyrazole;
(24) 4-(4-ethyl-benzyl)-1-(2-fluoro-1-fluoromethyl-ethyl)-5-trifluoromethyl-3-β-D-glucopyranos-1-yloxy-1H-pyrazole;
(25) 4-(3-fluoro-4-methoxy-benzyl)-1-isopropyl-5-trifluoromethyl-3-β-D-glucopyranos-1-yloxy-1H-pyrazole;
(26) 4-(3-fluoro-4-methyl-benzyl)-1-isopropyl-5-methyl-3-β-D-glucopyranos-1-yloxy-1H-pyrazole;
(27) 4-(2,3-difluoro-4-isopropoxy-benzyl)-1-isopropyl-5-methyl-3-β-D-glucopyranos-1-yloxy-1H-pyrazole;
(28) 4-(3-fluoro-4-methoxy-benzyl)-1-isopropyl-5-methyl-3-β-D-glucopyranos-1-yloxy-1H-pyrazole;
(29) 4-(4-ethyl-benzyl)-1-isopropyl-5-methyl-3-β-D-glucopyranos-1-yloxy-1H-pyrazole;

or a prodrug thereof wherein one or more hydroxyl groups of the β-D-glucopyranosyl group are acylated with groups selected from (C₁₋₃-alkyl)carbonyl, (C₁₋₆-alkyl)oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof;

in combination with at least one second therapeutic agent which is suitable in the treatment or prevention of one or more conditions selected from type 1 diabetes mellitus, type 2 diabetes mellitus, impaired glucose tolerance (IGT), impaired fasting blood glucose (IFG), and hyperglycemia.

According to another aspect of the invention there is provided a method for preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

According to another aspect of the invention there is provided a method for improving glycemic control and/or for reducing of fasting plasma glucose, of postprandial plasma glucose and/or of glycosylated hemoglobin HbA1c in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

The pharmaceutical composition according to this invention may also have valuable disease-modifying properties with respect to diseases or conditions related to impaired glucose tolerance (IGT), impaired fasting blood glucose (IFG), insulin resistance and/or metabolic syndrome.

According to another aspect of the invention there is provided a method for preventing, slowing, delaying or reversing progression from impaired glucose tolerance (IGT), impaired fasting blood glucose (IFG), insulin resistance and/or from metabolic syndrome to type 2 diabetes mellitus in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

As by the use of a pharmaceutical composition according to this invention an improvement of the glycemic control in patients in need thereof is obtainable, also those conditions and/or diseases related to or caused by an increased blood glucose level may be treated.

According to another aspect of the invention there is provided a method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter. The term “tissue ischaemia” particularly comprises diabetic macroangiopathy, diabetic microangiopathy, impaired wound healing and diabetic ulcer.

By the administration of a pharmaceutical composition according to this invention and due to the SGLT2 inhibitory activity of the pyrazole-O-glucoside derivative excessive blood glucose levels are not converted to insoluble storage forms, like fat, but excreted through the urine of the patient. Therefore no gain in weight or even a reduction of the weight is the result.

According to another aspect of the invention there is provided a method for reducing the weight or preventing an increase of the weight or facilitating a reduction of the weight in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

The pharmacological effect of the pyrazole-O-glucoside derivative in the pharmaceutical composition according to this invention is independent of insulin. Therefore an improvement of the glycemic control is possible without an additional strain on the pancreatic beta cells. By an administration of a pharmaceutical composition according to this invention a beta-cell degeneration and a decline of beta-cell functionality such as for example apoptosis or necrosis of pancreatic beta cells can be delayed or prevented. Furthermore the functionality of pancreatic cells can be improved or restored, and the number and size of pancreatic beta cells increased. It may be shown that the differentiation status and hyperplasia of pancreatic beta-cells disturbed by hyperglycemia can be normalized by treatment with a pharmaceutical composition according to this invention.

According to another aspect of the invention there is provided a method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

By the administration of a combination or pharmaceutical composition according to the present invention an abnormal accumulation of fat in the liver may be reduced or inhibited. Therefore according to another aspect of the present invention there is provided a method for preventing, slowing, delaying or treating diseases or conditions attributed to an abnormal accumulation of liver fat in a patient in need thereof characterized in that a pyrazole-O-glucoside derivative selected from the group of compounds (1) to (29) is administered in combination or alternation with at least one second therapeutic agent as defined hereinbefore and hereinafter. Diseases or conditions which are attributed to an abnormal accumulation of liver fat are particularly selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver or toxic fatty liver.

As a result thereof another aspect of the invention provides a method for maintaining and/or improving the insulin sensitivity and/or for treating or preventing hyperinsulinemia and/or insulin resistance in a patient in need thereof characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

According to another aspect of the invention there is provided the use of a pyrazole-O-glucoside derivative selected from the group of compounds (1) to (29) as defined hereinbefore and hereinafter for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing the weight or preventing an increase of the weight or facilitating a reduction of the weight; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver fat; or
maintaining and/or improving the insulin sensitivity and/or for treating or preventing hyperinsulinemia and/or insulin resistance;

in a patient in need thereof characterized in that the pyrazole-O-glucoside derivative is administered in combination or alternation with at least one second therapeutic agent as defined hereinbefore and hereinafter.

According to another aspect of the invention there is provided the use of at least one second therapeutic agent as defined hereinbefore and hereinafter for the manufacture of a medicament for
preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome; or
improving glycemic control and/or for reducing of fasting plasma glucose, of postprandial plasma glucose and/or of glycosylated hemoglobin HbA1c; or
preventing, slowing, delaying or reversing progression from impaired glucose tolerance (IGT), impaired fasting blood glucose (IFG), insulin resistance and/or from metabolic syndrome to type 2 diabetes mellitus; or
preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease; or
reducing the weight or preventing an increase of the weight or facilitating a reduction of the weight; or
preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion; or
preventing, slowing, delaying or treating diseases or conditions attributed to an abnormal accumulation of liver fat; or
maintaining and/or improving the insulin sensitivity and/or for treating or preventing hyperinsulinemia and/or insulin resistance;

in a patient in need thereof characterized in that the least one second therapeutic agent is administered in combination or alternation with a pyrazole-O-glucoside derivative selected from the group of compounds (1) to (29) as defined hereinbefore and hereinafter.

According to another aspect of the invention there is provided the use of a pharmaceutical composition according to the present invention for the manufacture of a medicament for a therapeutic and preventive method as described hereinbefore and hereinafter.

### DEFINITIONS

The term “active ingredient” of a pharmaceutical composition according to the present invention means the pyrazole-O-glucoside derivative and/or the second therapeutic ingredient.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference≧85 cm in men and ≧90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 110 mg/dL (6.11 mmol/L). The word “fasting” has the usual meaning as a medical term.

The term “hyperglycemia” is defined as the condition in which a subject has a fasting blood glucose concentration above the normal range, greater than 110 mg/dL (6.11 mmol/L). The word “fasting” has the usual meaning as a medical term.

The term “hypoglycemia” is typically defined as a condition in which a subject has symptoms known to be caused by hypoglycaemia, i.e. low blood glucose concentration at the time the symptoms occur and reversal or improvement of symptoms or problems when the blood glucose concentration is restored to normal. Typically, plasma glucose levels below 70 mg/dl (3.9 mmol/L), in particular below 60 mg/dl (3.3 mmol/L), are considered hypoglycaemic.

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration greater than 110 mg/dL and less than 126 mg/dl (7.00 mmol/L).

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast.

The term “hyperinsulinemia” is defined as the condition in which a subject with insulin resistance, with or without euglycemia, in which the fasting or postprandial serum or plasma insulin concentration is elevated above that of normal, lean individuals without insulin resistance, having a waist-to-hip ration<1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. In this method it is not possible to distinguish between hepatic and peripheral insulin resistance.

Furthermore insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl.1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):

HOMA-IR=[fasting serum insulin (μU/mL)]×[fasting plasma glucose (mmol/L)/22.5]

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected this is a particularly strong indication of the presence of prediabetes. Thus, it may be that in order to maintain glucose homoeostasis a person needs 2-3 times as much insulin as another person, without this having any direct pathological significance.

The methods to investigate the function of pancreatic beta-cells are similar to the above methods with regard to insulin sensitivity, hyperinsulinemia or insulin resistance: An improvement of the beta-cell function can be measured for example by determining a HOMA-index for beta-cell function (Matthews et al., Diabetologia 1985, 28: 412-19), the ratio of intact proinsulin to insulin (Forst et al., Diabetes 2003, 52(Suppl.1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “pre-diabetes” is the condition wherein an individual is pre-disposed to the development of type 2 diabetes. Pre-diabetes extends the definition of impaired glucose tolerance to include individuals with a fasting blood glucose within the high normal range≧100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes. Insulin resistance can be confirmed in these individuals by calculating HOMA-IR score. For the purpose of this invention, insulin resistance is defined as the clinical condition in which an individual has a HOMA-IR score>4.0 or a HOMA-IR score above the upper limit of normal as defined for the laboratory performing the glucose and insulin assays.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example metformin on its own achieves an average improvement in the HbA1c value in the diabetic of the order of 1.0-1.5% %. This reduction of the HbA1c value is not sufficient in all diabetics to achieve the desired target range of <6.5% and preferably <6% HbA1c.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference>40 inches or 102 cm in men, and >35 inches or 94 cm in women; or with regard to a Japanese ethnicity or Japanese patients defined as waist circumference≧85 cm in men and ≧90 cm in women;
2. Triglycerides: ≧150 mg/dL
3. HDL-cholesterol<40 mg/dL in men
4. Blood pressure≧130/85 mm Hg (SBP≧130 or DBP≧85)
5. Fasting blood glucose≧110 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor and Diagnose”, TH-Books Verlagsgesellschaft mbH, Frankfurt/Main, 2000.

According to a commonly used definition hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The terms “prophylactically treating” and “preventing” are used interchangeably.

### DETAILED DESCRIPTION

The aspects according to the present invention, in particular the pharmaceutical compositions, methods and uses, refer to pyrazole-O-glucoside derivatives selected from the group of compounds (1) to (29) as defined hereinbefore and hereinafter, or prodrugs thereof, or pharmaceutically acceptable salts thereof.

Preferably all hydroxyl groups are not substituted or only the hydroxyl group connected to the carbon atom at the 6^{th} position of the β-D-glucopyranosyl group is substituted as defined. Preferred substituents are selected from among (C₁₋₃-alkyl)carbonyl, (C₁₋₆-alkyl)oxycarbonyl, phenyloxycarbonyl, benzyloxycarbonyl and benzylcarbonyl. Even more preferred substituents are selected from among acetyl, methoxycarbonyl and ethoxycarbonyl, in particular ethoxycarbonyl.

Preferred prodrugs are selected from the group consisting of
(30a) 4-(2,3-difluoro-4-methoxy-benzyl)-1-isopropyl-5-methyl-3-(6-O-methoxycarbonyl-β-D-glucopyranos-1-yloxy)-1H-pyrazole;
(30b) 4-(2,3-difluoro-4-methoxy-benzyl)-1-isopropyl-5-methyl-3-(6-O-ethoxycarbonyl-β-D-glucopyranos-1-yloxy)-1H-pyrazole;
(31a) 4-(3-fluoro-4-ethoxy-benzyl)-1-isopropyl-5-methyl-3-(6-O-methoxycarbonyl-β-D-glucopyranos-1-yloxy)-1H-pyrazole;
(31b) 4-(3-fluoro-4-ethoxy-benzyl)-1-isopropyl-5-methyl-3-(6-O-ethoxycarbonyl-β-D-glucopyranos-1-yloxy)-1H-pyrazole;
(32a) 4-(3-fluoro-4-isopropoxy-benzyl)-1-isopropyl-5-methyl-3-(6-O-methoxycarbonyl-β-D-glucopyranos-1-yloxy)-1H-pyrazole;
(32b) 4-(3-fluoro-4-isopropoxy-benzyl)-1-isopropyl-5-methyl-3-(6-O-ethoxycarbonyl-β-D-glucopyranos-1-yloxy)-1H-pyrazole;
(33a) 4-(2,5-difluoro-4-methoxy-benzyl)-1-isopropyl-5-methyl-3-(6-O-methoxycarbonyl-β-D-glucopyranos-1-yloxy)-1H-pyrazole;
(33b) 4-(2,5-difluoro-4-methoxy-benzyl)-1-isopropyl-5-methyl-3-(6-O-ethoxycarbonyl-β-D-glucopyranos-1-yloxy)-1H-pyrazole;
(34a) 4-(2-fluoro-4-isopropoxy-benzyl)-1-isopropyl-5-methyl-3-(6-O-methoxycarbonyl-β-D-glucopyranos-1-yloxy)-1H-pyrazole;
(34b) 4-(2-fluoro-4-isopropoxy-benzyl)-1-isopropyl-5-methyl-3-(6-O-ethoxycarbonyl-β-D-glucopyranos-1-yloxy)-1H-pyrazole;
(35a) 4-(2-fluoro-4-ethoxy-benzyl)-1-isopropyl-5-methyl-3-(6-O-methoxycarbonyl-β-D-glucopyranos-1-yloxy)-1H-pyrazole;
(35b) 4-(2-fluoro-4-ethoxy-benzyl)-1-isopropyl-5-methyl-3-(6-O-ethoxycarbonyl-β-D-glucopyranos-1-yloxy)-1H-pyrazole;
(36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole;
(36b) 4-(2-fluoro-4-methoxy-benzyl)-1-isopropyl-5-trifluoromethyl-3-(6-O-ethoxycarbonyl-β-D-glucopyranos-1-yloxy)-1H-pyrazole;
(37a) 4-(2,6-difluoro-4-methoxy-benzyl)-1-isopropyl-5-methyl-3-(6-O-methoxycarbonyl-β-D-glucopyranos-1-yloxy)-1H-pyrazole;
(37b) 4-(2,6-difluoro-4-methoxy-benzyl)-1-isopropyl-5-methyl-3-(6-O-ethoxycarbonyl-β-D-glucopyranos-1-yloxy)-1H-pyrazole;
(38a) 4-(3,5-difluoro-4-methoxy-benzyl)-1-isopropyl-5-methyl-3-(6-O-methoxycarbonyl-β-D-glucopyranos-1-yloxy)-1H-pyrazole;
(38b) 4-(3,5-difluoro-4-methoxy-benzyl)-1-isopropyl-5-methyl-3-(6-O-ethoxycarbonyl-β-D-glucopyranos-1-yloxy)-1H-pyrazole;
(39a) 1-cyclobutyl-4-(3-fluoro-4-methyl-benzyl)-5-methyl-3-(6-O-methoxycarbonyl-β-D-glucopyranos-1-yloxy)-1H-pyrazole;
(39b) 1-cyclobutyl-4-(3-fluoro-4-methyl-benzyl)-5-methyl-3-(6-O-ethoxycarbonyl-β-D-glucopyranos-1-yloxy)-1H-pyrazole;
(40a) 1-cyclopropylmethyl-4-(3-fluoro-4-methyl-benzyl)-5-methyl-3-(6-methoxycarbonyl-β-D-glucopyranos-1-yloxy)-1H-pyrazole;
(40b) 1-cyclopropylmethyl-4-(3-fluoro-4-methyl-benzyl)-5-methyl-3-(6-ethoxycarbonyl-β-D-glucopyranos-1-yloxy)-1H-pyrazole;
(41a) 1-cyclobutyl-4-(2-fluoro-4-methoxy-benzyl)-5-methyl-3-(6-O-methoxycarbonyl-β-D-glucopyranos-1-yloxy)-1H-pyrazole;
(41b) 1-cyclobutyl-4-(2-fluoro-4-methoxy-benzyl)-5-methyl-3-(6-O-ethoxycarbonyl-β-D-glucopyranos-1-yloxy)-1H-pyrazole;
(42a) 4-(4-bromo-3-fluoro-benzyl)-1-isopropyl-5-methyl-3-(6-O-methoxycarbonyl-β-D-glucopyranos-1-yloxy)-1H-pyrazole;
(42b) 4-(4-bromo-3-fluoro-benzyl)-1-isopropyl-5-methyl-3-(6-O-ethoxycarbonyl-β-D-glucopyranos-1-yloxy)-1H-pyrazole;
(43a) 4-(2,3-difluoro-4-methyl-benzyl)-1-isopropyl-5-methyl-3-(6-O-methoxycarbonyl-β-D-glucopyranos-1-yloxy)-1H-pyrazole;
(43b) 4-(2,3-difluoro-4-methyl-benzyl)-1-isopropyl-5-methyl-3-(6-O-ethoxycarbonyl-β-D-glucopyranos-1-yloxy)-1H-pyrazole;
(44a) 4-(4-bromo-2-fluoro-benzyl)-1-isopropyl-5-methyl-3-(6-O-methoxycarbonyl-β-D-glucopyranos-1-yloxy)-1H-pyrazole;
(44b) 4-(4-bromo-2-fluoro-benzyl)-1-isopropyl-5-methyl-3-(6-O-ethoxycarbonyl-β-D-glucopyranos-1-yloxy)-1H-pyrazole;
(45a) 4-(2-fluoro-4-methyl-benzyl)-1-isopropyl-5-methyl-3-(6-O-methoxycarbonyl-β-D-glucopyranos-1-yloxy)-1H-pyrazole;
(45b) 4-(2-fluoro-4-methyl-benzyl)-1-isopropyl-5-methyl-3-(6-O-ethoxycarbonyl-β-D-glucopyranos-1-yloxy)-1H-pyrazole;

or pharmaceutically acceptable salts thereof.

In addition further preferred prodrugs are selected from the group consisting of the compounds (46) to (63),
(46) 4-(3-Fluoro-4-methyl-benzyl)-1-isopropyl-5-methyl-3-(6-O-ethoxycarbonyl-β-D-glucopyranos-1-yloxy)-1H-pyrazole;
(47) 4-(2-Fluoro-4-methoxy-benzyl)-1-isopropyl-5-methyl-3-(6-O-ethoxycarbonyl-β-D-glucopyranos-1-yloxy)-1H-pyrazole;
(48) 4-(2-Fluoro-4-methoxy-benzyl)-1-isopropyl-5-methyl-3-(6-O-isobutyloxycarbonyl-β-D-glucopyranos-1-yloxy)-1H-pyrazole;
(49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole;
(50) 4-(2-Fluoro-4-methoxy-benzyl)-1-isopropyl-5-methyl-3-(6-O-phenoxycarbonyl-β-D-glucopyranos-1-yloxy)-1H-pyrazole;
(51) 4-(2-Fluoro-4-methoxy-benzyl)-1-isopropyl-5-methyl-3-(6-O-benzyloxycarbonyl-β-D-glucopyranos-1-yloxy)-1H-pyrazole;
(52) 4-(2-Fluoro-4-methoxy-benzyl)-1-isopropyl-5-methyl-3-(6-O-acetyl-β-D-glucopyranos-1-yloxy)-1H-pyrazole;
(53) 4-(2-Fluoro-4-methoxy-benzyl)-1-isopropyl-5-methyl-3-(6-O-propylcarbonyl-β-D-glucopyranos-1-yloxy)-1H-pyrazole;
(54) 4-(2-Fluoro-4-methoxy-benzyl)-1-isopropyl-5-methyl-3-(6-O-isopropylcarbonyl-β-D-glucopyranos-1-yloxy)-1H-pyrazole;
(55) 4-(2-Fluoro-4-methoxy-benzyl)-1-isopropyl-5-methyl-3-(6-O-benzylcarbonyl-β-D-glucopyranos-1-yloxy)-1H-pyrazole;
(56) 4-(4-ethyl-benzyl)-1-isopropyl-5-trifluoromethyl-3-(6-O-ethoxycarbonyl-β-D-glucopyranos-1-yloxy)-1H-pyrazole;
(57) 4-(4-bromo-benzyl)-1-isopropyl-5-trifluoromethyl-3-(6-O-ethoxycarbonyl-β-D-glucopyranos-1-yloxy)-1H-pyrazole;
(58) 4-(4-ethyl-benzyl)-1-cyclobutyl-5-trifluoromethyl-3-(6-O-ethoxycarbonyl-β-D-glucopyranos-1-yloxy)-1H-pyrazole;
(59) 4-(4-ethyl-benzyl)-1-(2-fluoro-1-fluoromethyl-ethyl)-5-trifluoromethyl-3-(6-O-ethoxycarbonyl-β-D-glucopyranos-1-yloxy)-1H-pyrazole;
(60) 4-(3-fluoro-4-methoxy-benzyl)-1-isopropyl-5-trifluoromethyl-3-(6-O-ethoxycarbonyl-β-D-glucopyranos-1-yloxy)-1H-pyrazole;
(61) 4-(4-isopropoxy-benzyl)-1-isopropyl-5-methyl-3-(6-O-ethoxycarbonyl-β-D-glucopyranos-1-yloxy)-1H-pyrazole;
(62) 4-(3-fluoro-4-methoxy-benzyl)-1-isopropyl-5-methyl-3-(6-O-ethoxycarbonyl-β-D-glucopyranos-1-yloxy)-1H-pyrazole;
(63) 4-(4-ethyl-benzyl)-1-isopropyl-5-methyl-3-(6-O-ethoxycarbonyl-β-D-glucopyranos-1-yloxy)-1H-pyrazole;

or pharmaceutically acceptable salts thereof, as defined hereinbefore and hereinafter.

Yet further preferred prodrugs are selected from the group consisting of the compounds (64) to (73)
(64) 4-(3-fluoro-4-methoxy-benzyl)-1-isopropyl-5-methyl-3-(6-O-methoxycarbonyl-β-D-glucopyranos-1-yloxy)-1H-pyrazole;
(65) 4-(4-ethyl-benzyl)-1-isopropyl-5-trifluoromethyl-3-(6-O-methoxycarbonyl-β-D-glucopyranos-1-yloxy)-1H-pyrazole;
(66) 4-(4-bromo-benzyl)-1-isopropyl-5-trifluoromethyl-3-(6-O-methoxycarbonyl-β-D-glucopyranos-1-yloxy)-1H-pyrazole;
(67) 4-(4-ethyl-benzyl)-1-cyclobutyl-5-trifluoromethyl-3-(6-O-methoxycarbonyl-β-D-glucopyranos-1-yloxy)-1H-pyrazole;
(68) 4-(4-ethyl-benzyl)-1-(2-fluoro-1-fluoromethyl-ethyl)-5-trifluoromethyl-3-(6-O-methoxycarbonyl-β-D-glucopyranos-1-yloxy)-1H-pyrazole;
(69) 4-(3-fluoro-4-methoxy-benzyl)-1-isopropyl-5-trifluoromethyl-3-(6-O-methoxycarbonyl-β-D-glucopyranos-1-yloxy)-1H-pyrazole;
(70) 4-(3-fluoro-4-methyl-benzyl)-1-isopropyl-5-methyl-3-(6-O-methoxycarbonyl-β-D-glucopyranos-1-yloxy)-1H-pyrazole;
(71) 4-(4-isopropoxy-benzyl)-1-isopropyl-5-methyl-3-(6-O-methoxycarbonyl-β-D-glucopyranos-1-yloxy)-1H-pyrazole;
(72) 4-(2-fluoro-4-methoxy-benzyl)-1-isopropyl-5-methyl-3-(6-O-methoxycarbonyl-β-D-glucopyranos-1-yloxy)-1H-pyrazole;
(73) 4-(4-ethyl-benzyl)-1-isopropyl-5-methyl-3-(6-O-methoxycarbonyl-β-D-glucopyranos-1-yloxy)-1H-pyrazole;

or a pharmaceutically acceptable salt thereof.

According to a first preferred embodiment the aspects according to the present invention, in particular the pharmaceutical compositions, the methods and uses, refer to
(1) 4-(2,3-difluoro-4-methoxy-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (30a) and (30b).

According to a second preferred embodiment the aspects according to the present invention, in particular the pharmaceutical compositions, the methods and uses, refer to
(11) 4-(2,3-difluoro-4-methyl-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (43a) and (43b).

According to a third preferred embodiment the aspects according to the present invention, in particular the pharmaceutical compositions, the methods and uses, refer to
(12) 4-(2-fluoro-4-methyl-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (45a) and (45b).

According to a fourth preferred embodiment the aspects according to the present invention, in particular the pharmaceutical compositions, the methods and uses, refer to
(16) 4-(2-fluoro-4-methoxy-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (47) and (72).

According to a fifth preferred embodiment the aspects according to the present invention, in particular the pharmaceutical compositions, the methods and uses, refer to
(20) 4-(2-fluoro-4-ethoxy-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (35a) and (35b).

According to a sixth preferred embodiment the aspects according to the present invention, in particular the pharmaceutical compositions, the methods and uses, refer to
(26) 4-(3-fluoro-4-methyl-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (46) and (70).

According to a seventh preferred embodiment the aspects according to the present invention, in particular the pharmaceutical compositions, the methods and uses, refer to
(28) 4-(3-fluoro-4-methoxy-benzyl)-1-isopropyl-5-methyl-3-β-D-glucopyranos-1-yloxy-1H-pyrazole;

or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (62) and (64).

The aspects according to the present invention, in particular the pharmaceutical compositions, methods and uses, refer to an at least one therapeutic agent which is suitable in the treatment or prevention of one or more conditions selected from type 1 diabetes mellitus, type 2 diabetes mellitus, impaired glucose tolerance (IGT), impaired fasting blood glucose (IFG) and hyperglycemia.

Preferably the at least one second therapeutic agent is selected from the groups a) to k) consisting of:

a) biguanides,

b) sulfonylureas,

c) metiglinides,

d) thiazolidindiones,

e) alpha-glucosidase inhibitors,

f) insulins and insulin analogues,

g) GLP1 and GLP1 analogues,

h) PPAR gamma modulators,

i) PPAR gamma/alpha modulators,

j) glucose-dependent insulinotropic polypeptide agonists,

k) beta-3 agonists, and

l) dipeptidyl peptidase IV inhibitors (DPP IV inhibitors).

More preferably the at least one second therapeutic agent is selected from the groups a) to f) as described hereinbefore and hereinafter.

Examples of biguanides are metformin, phenformin and buformin. A pyrazole-O-glucoside in combination with a biguanide, for example with metformin, can improve glycemic control and may act synergistically with the biguanide, for example to reduce weight that has overall beneficial effects on the metabolic syndrome which is commonly associated with type 2 diabetes mellitus.

Examples of sulfonylureas are glibenclamide, tolbutamide, glimepiride, glipizide, gliquidone, glibornurid, glyburide and gliclazide. As the efficacy of sulfonylureas wears off over the course of treatment, a combination of a pyrazole-O-glucoside with a sulfonylurea may offer additional benefit to the patient in terms of better glycemic control. Also, treatment with sulfonylureas is normally associated with gradual weight gain over the course of treatment and weight reducing capability of a pyrazole-O-glucoside can minimize this side effect of the treatment with an sulfonylurea and improve the metabolic syndrome. This combination may also allow a reduction in the dose of sulfonylureas which may translate into less hypoglycemia which is an undesirable side effect of sulfonylureas.

Examples of meglitinides are nateglinide, repaglinide and mitiglinide. As the efficacy of sulfonylureas wears off over the course of treatment, a combination of a pyrazole-O-glucoside with a meglitinide may offer additional benefit to the patient in terms of better glycemic control. Also, treatment with meglitinides is normally associated with gradual weight gain over the course of treatment and weight reducing capability of a pyrazole-O-glucoside can minimize this side effect of the treatment with an meglitinide and improve the metabolic syndrome. This combination may also allow a reduction in the dose of meglitinides which may translate into less hypoglycemia which is an undesirable side effect of meglitinides.

Examples of thiazolidindiones are pioglitazone, rosiglitazone, troglitazone and ciglitazone. Additional benefits from the combination of a pyrazole-O-glucoside and a thiazolidindione may relate to synergistic reduction in blood glucose, an improved glycemic control, an improvement of fluid retention caused by thiazolidindiones and reducing or nullifying weight gain associated with the use of thiazolidindiones.

Examples of alpha-glucosidase blockers (alpha-glucosidase inhibitors) are miglitol, acarbose and voglibose. A combination of a pyrazole-O-glucoside and an alpha-glucosidase inhibitor will add to their blood glucose lowering effect and may allow a reduction in the dose of the alpha-glucosidase inhibitor that are commonly associated with unpleasant gastro-intestinal side effects, thereby making it more tolerable and improve the patients compliance with the treatment.

Examples of insulins and insulin analogues are short acting insulins like insulin lispro (Humalog®), insulin aspartat (Novorapid®), insulin glulisine (Apidra®), regular insulin, intermediate acting insulins like NPH-insulins and long acting insulins like lente and ultralente insulin, insulin glargine (Lantus®), insulin detemir (Levemir®). The term insulins includes recombinant insulins. The use of insulin is commonly associated with weight gain as a result of the anabolic effects of insulin as well as fluid retention. Combining a pyrazole-O-glucoside with insulin or an insulin analogue will achieve a better glycemic control with lower doses of insulin. Given the mechanism of action of the pyrazole-O-glucoside, such a combination is likely to ameliorate the fluid retention and edema associated with insulin use.

An example of GLP1 and GLP1 analogues is exendin-4 (exenatide). Combining a pyrazole-O-glucoside with a GLP-1 analogue is expected to improve glycemic control and add to GLP-1 analogue weight reducing effect.

An example of PPAR gamma modulators is metaglidasen. Combining a pyrazole-O-glucoside with a PPAR gamma modulator is expected to improve glycemic control.

Examples of PPAR gamma/alpha modulators are tesaglitazar, muraglitazar and KRP297. Combining a pyrazole-O-glucoside with a PPAR gamma/alpha modulator is expected to improve glycemic control.

Examples of glucose-dependent insulinotropic polypeptide agonists are pramlintide and amlyin. Combinations with such second therapeutic agents are expected to improve glycemic control.

Examples of beta-3 agonists are ritobegron, YM 178, solabegron, talibegron, N-5984, GRC-1087, rafabegron and FMP825. Combining a pyrazole-O-glucoside with a beta-3 agonist is expected to improve glycemic control.

Examples of DPP IV inhibitors are sitagliptin, vildagliptin, saxagliptin and alogliptin.

Even more preferably the at least one second therapeutic agent is selected from the group consisting of metformin, glibenclamide, tolbutamide, glimepiride, glipizid, gliquidon, glibornurid, gliclazid, nateglinide, repaglinide, pioglitazone, rosiglitazone, miglitol, voglibose, acarbose, insulins and insulin analogues, in particular short and long acting insulins, metaglidasen and pramlintide.

Most preferably the at least one second therapeutic agent is selected from the group consisting of metformin, glimepiride, pioglitazone, rosiglitazone, miglitol, voglibose, acarbose, insulins and insulin analogues, in particular short and long acting insulins.

According to this invention it is to be understood that the definitions of the above listed second therapeutic agents also comprise their pharmaceutically acceptable salts as well as hydrates, solvates and polymorphic forms thereof.

Most preferably the pyrazole-O-glucoside is selected according to the 1^{st}, 2^{nd}, 3^{rd}, 4^{th}, 5^{th} or 6^{th} embodiment as described hereinbefore and the second therapeutic agent is selected from the groups of preferred agents as described hereinbefore.

Therefore the pharmaceutical compositions, methods and uses according to this invention most preferably relate to combinations which are selected from the Table 1.

TABLE 1

| | Pyrazole-O-glucoside | |
|---|---|---|
| | derivative | |
| | selected according | |
| No. | to the embodiment No. | Second therapeutic agent |
| 1a | 1^{st} | metformin |
| 1b | 1^{st} | glimepiride |
| 1c | 1^{st} | pioglitazone |
| 1d | 1^{st} | rosiglitazone |
| 1e | 1^{st} | miglitol |
| 1f | 1^{st} | insulins and insulin analogues, in |
| | | particular short and/or long acting insulins |
| 1g | 1^{st} | acarbose |
| 1h | 1^{st} | voglibose |
| 2a | 2^{nd} | metformin |
| 2b | 2^{nd} | glimepiride |
| 2c | 2^{nd} | pioglitazone |
| 2d | 2^{nd} | rosiglitazone |
| 2e | 2^{nd} | miglitol |
| 2f | 2^{nd} | insulins and insulin analogues, in |
| | | particular short and/or long acting insulins |
| 2g | 2^{nd} | acarbose |
| 2h | 2^{nd} | voglibose |
| 3a | 3^{rd} | metformin |
| 3b | 3^{rd} | glimepiride |
| 3c | 3^{rd} | pioglitazone |
| 3d | 3^{rd} | rosiglitazone |
| 3e | 3^{rd} | miglitol |
| 3f | 3^{rd} | insulins and insulin analogues, in |
| | | particular short and/or long acting insulins |
| 3g | 3^{rd} | acarbose |
| 3h | 3^{rd} | voglibose |
| 4a | 4^{th} | metformin |
| 4b | 4^{th} | glimepiride |
| 4c | 4^{th} | pioglitazone |
| 4d | 4^{th} | rosiglitazone |
| 4e | 4^{th} | miglitol |
| 4f | 4^{th} | insulins and insulin analogues, in |
| | | particular short and/or long acting insulins |
| 4g | 4^{th} | acarbose |
| 4h | 4^{th} | voglibose |
| 5a | 5^{th} | metformin |
| 5b | 5^{th} | glimepiride |
| 5c | 5^{th} | pioglitazone |
| 5d | 5^{th} | rosiglitazone |
| 5e | 5^{th} | miglitol |
| 5f | 5^{th} | insulins and insulin analogues, in |
| | | particular short and/or long acting insulins |
| 5g | 5^{th} | acarbose |
| 5h | 5^{th} | voglibose |
| 6a | 6^{th} | metformin |
| 6b | 6^{th} | glimepiride |
| 6c | 6^{th} | pioglitazone |
| 6d | 6^{th} | rosiglitazone |
| 6e | 6^{th} | miglitol |
| 6f | 6^{th} | insulins and insulin analogues, in |
| | | particular short and/or long acting insulins |
| 6g | 6^{th} | acarbose |
| 6h | 6^{th} | voglibose |
| 7a | 7^{th} | metformin |
| 7b | 7^{th} | glimepiride |
| 7c | 7^{th} | pioglitazone |
| 7d | 7^{th} | rosiglitazone |
| 7e | 7^{th} | miglitol |
| 7f | 7^{th} | insulins and insulin analogues, in |
| | | particular short and/or long acting insulins |
| 7g | 7^{th} | acarbose |
| 7h | 7^{th} | voglibose |

When this invention refers to patients requiring treatment or prevention, it relates primarily to treatment and prevention in humans, but the pharmaceutical composition may also be used accordingly in veterinary medicine on mammals.

As described hereinbefore by the administration of the pharmaceutical composition according to this invention and in particular in view of the SGLT2 inhibitors activity of the pyrazole-O-glucoside derivative therein, excessive blood glucose is excreted through the urine of the patient, so that no gain in weight or even a reduction of the weight may result. Therefore a treatment or prophylaxis according to this invention is advantageously suitable in those patients in need of such treatment or prophylaxis who are diagnosed of one or more of the conditions selected from the group consisting of overweight, class I obesity, class II obesity, class III obesity, visceral obesity and abdominal obesity or for those individuals in which a weight increase is contraindicated.

The pharmaceutical composition according to this invention and in particular the pyrazole-O-glucoside derivative therein exhibits a very good efficacy with regard to glycemic control, in particular in view of a reduction of fasting plasma glucose, postprandial plasma glucose and/or glycosylated hemoglobin (HbA1c). By administering a pharmaceutical composition according to this invention, a reduction of HbA1c equal to or greater than preferably 0.5%, even more preferably equal to or greater than 1.0% can be achieved and the reduction is particularly in the range from 1.0% to 1.5%.

Furthermore the method and/or use according to this invention is advantageously applicable in those patients who show one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%.

The present invention also discloses the use of the pharmaceutical composition for improving glycemic control in patients having type 2 diabetes or showing first signs of prediabetes. Thus, the invention also includes diabetes prevention. If therefore a pharmaceutical composition according to this invention is used to improve the glycemic control as soon as one of the above-mentioned signs of prediabetes is present, the onset of manifest type 2 diabetes mellitus can be delayed or prevented.

Furthermore the pharmaceutical composition according to this invention is particularly suitable in the treatment of patients with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

It can be found that by using a pharmaceutical composition according to this invention, or a prodrug or pharmaceutically acceptable salt thereof, an improvement of the glycemic control can be achieved even in those patients who have insufficient glycemic control in particular despite treatment with an antidiabetic drug, for example despite maximal tolerated dose of oral monotherapy with either metformin or an antidiabetic of the class of sulphonylureas. A maximal tolerated dose with regard to metformin is for example 850 mg three times a day or any equivalent thereof. In the scope of the present invention the term “insufficient glycemic control” means a condition wherein patients show HbA1c values above 6.5%, in particular above 8%.

Therefore according to a preferred embodiment of the present invention there is provided a method for improving glycemic control and/or for reducing of fasting plasma glucose, of postprandial plasma glucose and/or of glycosylated hemoglobin HbA1c in a patient in need thereof who is diagnosed with impaired glucose tolerance (IGT), impaired fasting blood glucose (IFG) with insulin resistance, with metabolic syndrome and/or with type 2 or type 1 diabetes mellitus characterized in that a pyrazole-O-glucoside derivative selected from the group of compounds (1) to (29) as defined hereinbefore and hereinafter is administered in combination or alternation with at least one second therapeutic agent as defined hereinbefore and hereinafter.

The lowering of the blood glucose level by the administration of a pyrazole-O-glucoside derivative according to this invention, or a prodrug or pharmaceutically acceptable salt thereof, is insulin-independent. Therefore a pharmaceutical composition according to this invention is particularly suitable in the treatment of patients who are diagnosed having one or more of the following conditions
insulin resistance,
hyperinsulinemia,
pre-diabetes,
type 2 diabetes mellitus, particular having a late stage type 2 diabetes mellitus,
type 1 diabetes mellitus.

Furthermore a pharmaceutical composition according to this invention is particularly suitable in the treatment of patients who are diagnosed having one or more of the following conditions
(a) obesity (including class I, II and/or III obesity), visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL.

It is assumed that patients diagnosed with impaired glucose tolerance (IGT), impaired fasting blood glucose (IFG), with insulin resistance and/or with metabolic syndrome suffer from an increased risk of developing a cardiovascular disease, such as for example myocardial infarction, coronary heart disease, heart insufficiency, thromboembolic events. A glycemic control according to this invention may result in a reduction of the cardiovascular risks.

A pharmaceutical composition according to this invention, in particular due to the pyrazole-O-glucoside therein, exhibits a good safety profile. Therefore a treatment or prophylaxis according to this invention is advantageous possible in those patients for which the mono-therapy with another antidiabetic drug, such as for example metformin, is contraindicated and/or who have an intolerance against such drugs at therapeutic doses. In particular a treatment or prophylaxis according to this invention may be advantageous possible in those patients showing or having an increased risk for one or more of the following disorders: renal insufficiency or diseases, cardiac diseases, cardiac failure, hepatic diseases, pulmonal diseases, catabolytic states and/or danger of lactate acidosis, or female patients being pregnant or during lactation.

Furthermore, given that the excretion of glucose in urine as a result of SGLT-2 inhibition may cause mild diuresis (as seen in individuals with congenital SGLT-2 deficiency), the use of the pyrazole-O-glucoside derivative in combination with a second therapeutic agent as defined hereinbefore and hereinafter, which is known to cause fluid retention, for example insulin, an insulin analogues and/or a thiazolidindione, may be of special interest. This potential attribute may be particularly important in the management of type 2 diabetes mellitus in the acute phase of myocardial infarction. Such patients are susceptible to acute heart failure secondary to fluid retention as a result of the insulin therapy currently recommended for these patients in treatment guidelines. Therefore another aspect of the present invention relates to a method for the treatment of type 2 diabetes mellitus in a patient being in an acute phase of myocardial infarction by administering a pyrazole-O-glucoside derivative according to this invention in combination or alternation with at least one second therapeutic agent according to this invention, in particular with a thiazolidindione, such as pioglitazone, rosiglitazone, troglitazone or ciglitazone, or with insulin or with an insulin analogue.

Furthermore it can be found that the administration of a pharmaceutical composition according to this invention results in no risk or in a low risk of hypoglycemia. Therefore a treatment or prophylaxis according to this invention is also advantageously possible in those patients showing or having an increased risk for hypoglycemia.

A pharmaceutical composition according to this invention is particularly suitable in the long term treatment or prophylaxis of the diseases and/or conditions as described hereinbefore and hereinafter, in particular in the long term glycemic control in patients with type 2 diabetes mellitus.

The term “long term” as used hereinbefore and hereinafter indicates a treatment of or administration in a patient within a period of time longer than 12 weeks, preferably longer than 25 weeks, even more preferably longer than 1 year.

Therefore a particularly preferred embodiment of the present invention provides a method for therapy, preferably oral therapy, for improvement, especially long term improvement, of glycemic control in patients with type 2 diabetes mellitus, especially in patients with late stage type 2 diabetes mellitus, in particular in patients additionally diagnosed of overweight, obesity (including class I, class II and/or class III obesity), visceral obesity and/or abdominal obesity.

Administration of the pyrazole-O-glucoside derivative according to this invention in combination with with at least one second therapeutic agent can have an additive or over-additive effect and provide for dose reduction, side-effect reduction and/or interval extension when compared to the individual pyrazole-O-glucoside derivative or to the individual second therapeutic agent used in monotherapy in the usual way. The effects mentioned above are observed both when the pyrazole-O-glucoside derivative and the second therapeutic agent are administered in combination, for example simultaneously, and when they are administered in alternation, for example successively in separate formulations. In the case of the second therapeutic agent being an injectable, especially a biological agent, other benefits of a combination with the pyrazole-O-glucoside derivative may be seen, as for example, cost reduction by way of interval and/or dose reduction.

It will be appreciated that the amount of the pharmaceutical composition according to this invention to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician. In general however the pyrazole-O-glucoside derivative according to this invention, or the prodrug or pharmaceutically acceptable salt thereof, and the at least one second therapeutic agent are included in the pharmaceutical composition or dosage form in an amount sufficient that by their administration in combination or alternation the glycemic control in the patient to be treated is improved.

In the following preferred ranges of the amount of pyrazole-O-glucoside derivative and of the second therapeutic agent to be employed in the pharmaceutical composition and the methods and uses according to this invention are described. These ranges refer to the amounts to be administered per day with respect to an adult patient and can be adapted accordingly with regard to an administration 2, 3, 4 or more times daily and with regard to other routes of administration and with regard to the age of the patient.

Within the scope of the present invention the pharmaceutical composition (with the exception of insulin) is preferably administered orally. Other forms of administration are possible and described hereinafter. Preferably the dosage form comprising the pyrazole-O-glucoside is administered orally. The route of administration of the 2^{nd} therapeutic agent is usually well known.

In general the amount of the pyrazol-O-glucoside derivative in the pharmaceutical composition and methods according to this invention is preferably in the range from ⅕ to 1/1 of the amount usually recommended for a monotherapy using said pyrazole-O-glucoside derivative. Advantageously, the combination therapy according to the present invention utilizes lower dosages of the individual pyrazole-O-glucoside derivative or of the individual second therapeutic agent used in monotherapy or used in conventional therapeutics, thus avoiding possible toxicity and adverse side effects incurred when those agents are used as monotherapies.

The amount of the pyrazole-O-glucoside is preferably in the range from 1 mg to 1000 mg or 10 to 2000 mg, even more preferably from 10 to 500 mg or 50 to 800 mg, most preferably from 50 to 500 mg per day. The oral administration is preferred. Therefore a pharmaceutical composition may comprise the hereinbefore mentioned amounts for once daily administration and from 0.5 mg to 500 mg, even more preferably from 5 to 250 mg or 25 to 400 mg, most preferably from 25 to 250 mg for twice daily administration. An example is an amount of 200 mg or 400 mg of the pyrazole-O-glucoside according to the 1^{st}, 2^{nd}, 3^{rd}, 4^{th}, 5^{th}, 6^{th} or 7^{th} embodiment as described hereinbefore.

In general the amount of the second therapeutic agent in the pharmaceutical composition and methods according to this invention is preferably in the range from ⅕ to 1/1 of the amount usually recommended for a monotherapy using said second therapeutic agent.

A preferred dosage range of metformin is 100 to 3000 mg, in particular 200 to 2000 mg, most preferably 500 to 1000 per day. The preferred range of amounts in the pharmaceutical composition for an administration once, twice or three times daily is 100 to 3000, 50 to 1500 and 35 to 1000 mg respectively. Examples are 500 or 850 mg once, twice or three times daily, 1000 mg once or twice daily or 2000 mg once daily.

A preferred dosage range of pioglitazone is 5 to 50 mg per day. The preferred range of amounts in the pharmaceutical composition for an administration once, twice or three times daily is 5 to 50, 2 to 25 and 2 to 20 mg respectively. Examples are 15, 30 or 45 mg once daily.

A preferred dosage range of a rosiglitazone is 1 mg to 10 mg per day. The preferred range of amounts in the pharmaceutical composition for an administration once or twice times daily is 4 to 8 mg and 4 mg respectively.

A preferred dosage range of a thiazolidindione (other than pioglitazone or rosiglitazone as described above) is 2 to 100 mg per day. The preferred range of amounts in the pharmaceutical composition for an administration once, twice or three times daily is 2 to 100, 1 to 50 and 1 to 33 mg respectively.

A preferred dosage range of miglitol is 10 to 300 mg per day. The preferred range of amounts in the pharmaceutical composition for an administration once, twice or three times daily is 10 to 300, 5 to 150 and 3 to 100 mg respectively. Examples are 50 or 100 mg once, twice or three times daily.

A preferred dosage range of glibenclamide is 1 to 20 mg per day. The preferred range of amounts in the pharmaceutical composition for an administration once, twice or three times daily is 1 to 20, 0.5 to 10 and 0.5 to 7 mg respectively.

A preferred dosage range of tolbutamide is 100 to 3000 mg, preferably 500 to 3000 mg per day. The preferred range of amounts in the pharmaceutical composition for an administration once, twice or three times daily is 100 to 3000, 50 to 1500 and 35 to 1000 mg respectively.

A preferred dosage range of glimepiride is 0.5 to 10 mg, in particular 1 to 6 mg per day. The preferred range of amounts in the pharmaceutical composition for an administration once, twice or three times daily is 0.5 to 10, 0.25 to 5 and 0.2 to 3 mg respectively.

A preferred dosage range of glipizid is 1 to 50 mg, in particular 2.5 to 40 mg per day. The preferred range of amounts in the pharmaceutical composition for an administration once, twice or three times daily is 1 to 50, 0.5 to 25 and 0.3 to 17 mg respectively.

A preferred dosage range of gliquidon is 10 to 150 mg, in particular 30 to 120 mg per day. The preferred range of amounts in the pharmaceutical composition for an administration once, twice or three times daily is 10 to 150, 5 to 75 and 3 to 50 mg respectively.

A preferred dosage range of glibornurid is 5 to 75 mg per day. The preferred range of amounts in the pharmaceutical composition for an administration once, twice or three times daily is 5 to 75, 3 to 40 and 2 to 25 mg respectively.

A preferred dosage range of gliclazid is 25 to 320 mg, in particular 80 to 160 mg per day. The preferred range of amounts in the pharmaceutical composition for an administration once, twice or three times daily is 25 to 320, 12 to 160 and 10 to 80 mg respectively.

A preferred dosage range of nateglinide is 15 to 540 mg, in particular 60 to 360 mg per day. The preferred range of amounts in the pharmaceutical composition for an administration once, twice or three times daily is 15 to 360, 7 to 180 and 5 to 120 mg respectively.

A preferred dosage range of repaglinide is 0.1 to 16 mg, in particular 0.5 to 12 mg per day. The preferred range of amounts in the pharmaceutical composition for an administration once, twice or three times daily is 0.1 to 16, 0.05 to 8 and 0.03 to 5 mg respectively.

A preferred dosage range of metaglidasen is 40 to 600 mg, in particular 200 to 600 mg per day. The preferred range of amounts in the pharmaceutical composition for an administration once, twice or three times daily is 40 to 600, 20 to 300 and 15 to 200 mg respectively.

A preferred dosage range of a PPAR gamma/alpha modulator is 0.5 to 10 mg, in particular 2.5 to 5 mg per day. The preferred range of amounts in the pharmaceutical composition for an administration once, twice or three times daily is 0.5 to 10, 0.2 to 5 and 0.1 to 3 mg respectively.

A preferred dosage range of a pramlintide is 15 μg to 120 μg per day. The preferred range of amounts in the pharmaceutical composition for an administration once, twice or three times daily is 15 to 120, 8 to 60 and 5 to 40 μg respectively.

A preferred dosage range of an alpha glucosidase inhibitor is 0.1 to 500 mg per day. The preferred range of amounts in the pharmaceutical composition for an administration once, twice or three times daily is 0.1 to 500, 0.05 to 250 and 0.03 to 133 mg respectively.

A preferred dosage range of a voglibose is 0.1 to 2.0 mg per day, in particular 0.2 to 1.0 mg per day. The preferred range of amounts in the pharmaceutical composition for an administration twice or three times daily is 0.1 to 0.5 and 0.1 to 0.3 mg respectively.

A preferred dosage range of a acarbose is 50 to 300 mg per day, in particular 150 to 300 mg per day. The preferred range of amounts in the pharmaceutical composition for an administration twice or three times daily is 100 to 150 and 50 to 100 mg respectively. Examples are 50 or 100 mg twice or three times daily.

A preferred dosage range of a insulin is 1 to 250 IU per day. The preferred range of amounts in the pharmaceutical composition for an administration once, twice or three times daily is 1 to 250, 0.5 to 125 and 0.3 to 90 IU respectively. The term “IU” means international units.

The amount of the pyrazole-O-glucoside and of the second therapeutic agent in the pharmaceutical composition according to this invention correspond to the respective dosage ranges as provided hereinbefore. For example a pharmaceutical composition comprises an amount of 10 to 500 mg of an pyrazole-O-glucoside and metformin in an amount of 50 to 1500 mg.

In the methods and uses according to the present invention the pyrazole-O-glucoside derivative and the at least one second therapeutic ingredient are administered in combination or alternation. The term “administration in combination” means that both active ingredients are administered at the same time, i.e. simultaneously, or essentially at the same time. The term “administration in alternation” means that at first a first active ingredient is administered and after a period of time the second active ingredient is administered, i.e. both active ingredients are administered sequentially. The period of time may be in the range from 30 min to 12 hours. The administration which is in combination or in alternation may be once, twice, three times or four times daily.

With regard to the administration of the pyrazole-O-glucoside derivative in combination with the at least one second therapeutic ingredient both active ingredients may be present in a single dosage form, for example in a tablet or capsule, or each active ingredient may be present in a separate dosage form, for example in two different or identical dosage forms.

With regard to their administration in alternation each of the active ingredients is present in a separate dosage form, for example in two different or identical dosage forms.

Therefore the pharmaceutical composition according to this invention may be present as single dosage forms which comprise both the pyrazole-O-glucoside derivative and the at least one second therapeutic ingredient as well as separate dosage forms wherein one dosage form comprises the pyrazole-O-glucoside derivative and the other dosage form comprises the at least one second therapeutic ingredient.

The case may arise in which one active ingredient has to be administered more often, for example twice per day, than the other active ingredient, which for example needs administration once daily. Therefore the term “administration in combination or alternation” also includes an administration scheme in which first both active ingredients are administered in combination or alternation and after a period of time only one active ingredient is administered again or vice versa.

Therefore the present invention also includes pharmaceutical compositions which are present a separate dosage forms wherein one dosage form comprises the pyrazole-O-glucoside derivative and the second therapeutic agent and the other dosage form comprises either the the pyrazole-O-glucoside derivative or the at least one second therapeutic agent.

A pharmaceutical composition which is present as a separate or multiple dosage form, preferably as a kit of parts, is useful in combination therapy to flexibly suit the individual therapeutic needs of the patient.

A preferred kit of parts comprises
(a) a first containment containing a dosage form comprising the pyrazole-O-glucoside and at least one pharmaceutically acceptable carrier, and
(b) a second containment containing a dosage form comprising the at least one second therapeutic agent and at least one pharmaceutically acceptable carrier.

A further aspect of the present invention is a manufacture comprising the pharmaceutical composition being present as separate dosage forms according to the present invention and a label or package insert comprising instructions that the separate dosage forms are to be administered in combination or alternation.

A yet further aspect of the present invention is a manufacture comprising a medicament which comprises a pyrazole-O-glucoside derivative according to the present invention and a label or package insert which comprises instructions that the medicament may or is to be administered in combination or alternation with a medicament comprising at least one second therapeutic agent according to the present invention.

Another further aspect of the present invention is a manufacture comprising a medicament which comprises at least one second therapeutic agent according to the present invention and a label or package insert which comprises instructions that the medicament may or is to be administered in combination or alternation with a medicament comprising a pyrazole-O-glucoside derivative according to the present invention.

The desired dose of the pharmaceutical composition according to this invention may conveniently be presented in a once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers which must be “acceptable” in the sense of being compatible with the other ingredients of the formulation and not deleterious to the recipient thereof.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

The pharmaceutical compositions and methods according to this invention show advantageous effects in the treatment and prevention of those diseases and conditions as described hereinbefore compared with pharmaceutical compositions and methods which comprise only one of both active ingredients. Advantageous effect may be seen for example with respect to efficacy, dosage strength, dosage frequency, pharmacodynamic properties, pharmacokinetic properties, adverse effects, etc.

Any of the above mentioned combinations within the scope of the invention may be tested by animal models known in the art. In the following in vivo experiments are described which are suitable to evaluate pharmacologically relevant properties of pharmaceutical compositions and methods according to this invention:

Pharmaceutical compositions and methods according to this invention can be tested in genetically hyperinsulinemic or diabetic animals like db/db mice, ob/ob mice, Zucker Fatty (fa/fa) rats or Zucker Diabetic Fatty (ZDF) rats. In addition, they can be tested in animals with experimentally induced diabetes like HanWistar or Sprague Dawley rats pretreated with streptozotocin.

The effect on glycemic control of the combinations according to this invention can be tested after single or multiple dosing of a pyrazole-O-glucoside derivative and a second therapeutic agent alone and in combination in the animal models described hereinbefore by following the time course of blood glucose either in the fed state or after an oral glucose challenge in overnight fasted animals. The combinations according to the present invention significantly reduce glucose AUC or peak glucose concentrations compared to each monotherapy. In addition, after multiple dosing of a pyrazole-O-glucoside derivative and a second therapeutic agent alone and in combination in the animal models described hereinbefore, the effect on glycemic control can be determined by measuring the HbA1c value in blood. The combinations according to this invention significantly reduce HbA1c compared to each monotherapy.

The possible dose reduction of either the pyrazole-O-glucoside derivative or the second therapeutic agent or of both active ingredients can be tested by the effect on glycemic control of lower doses of the combinations and monotherapies in the animal models described hereinbefore. The combinations according to this invention at the lower doses significantly improve glycemic control compared to placebo treatment whereas the monotherapies at lower doses do not.

Examples of pharmaceutically acceptable carriers are known to the one skilled in the art. Methods for the manufacture of pyrazole-O-glucoside derivatives according to this invention and of prodrugs thereof are known to the one skilled in the art. Advantageously the compounds according to this invention can be prepared using synthetic methods as described in the literature, in particular as described in the EP 1 338 603 A1, EP 1 389 621 A1, WO 04/014932, WO 04/018491, WO 04/019958, WO 04/031203, WO 04/050122 and WO 03/020737. Preferred methods for the synthesis of the compounds according to this invention are described in the examples.

The methods of synthesis for the second therapeutic agent are described in the scientific literature and/or in published patent documents.

The pyrazole-O-glucoside and/or the second therapeutic agent may be present in the form of a pharmaceutically acceptable salt. Pharmaceutically acceptable salts include such as salts of inorganic acid like hydrochloric acid, sulfuric acid and phosphoric acid; salts of organic carboxylic acid like oxalic acid, acetic acid, citric acid, malic acid, benzoic acid, maleic acid, fumaric acid, tartaric acid, succinic acid and glutamic acid and salts of organic sulfonic acid like methanesulfonic acid and p-toluenesulfonic acid. The salts can be formed by combining the compound and an acid in the appropriate amount and ratio in a solvent and decomposer. They can be also obtained by the cation or anion exchange from the form of other salts.

The pyrazole-O-glucoside and/or the second therapeutic agent or a pharmaceutically acceptable salt thereof may be present in the form of a solvate such as a hydrate or alcohol adduct.

The biological properties of a pyrazole-O-glucoside derivative may be investigated as it is described for example in EP 1 338 603 A1, in particular with regard to the inhibiting activity on renal brush border membrane glucose uptake and to the activity on rat's sugar urine excretion. Furthermore the test as described in WO 05/021566 may be applied.

In the foregoing and following text, H atoms of hydroxyl groups are not explicitly shown in every case in structural formulae. The Examples that follow are intended to illustrate the present invention without restricting it.

### Pharmacological Examples

The following examples shows the beneficial effect on glycemic control of the combination of a pyrazole-O-glucoside and metformin according to the present invention as compared to the respective monotherapies. All experimental protocols concerning the use of laboratory animals are reviewed by a federal Ethics Committee and approved by governmental authorities. Male Zucker Diabetic Fatty (ZDF) rats (ZDF/Crl-Lepr^{fa}) at the age of 12 weeks are treated for 5 weeks with the pyrazole-O-glucoside (16), i.e. 4-(2-fluoro-4-methoxy-benzyl)-1-isopropyl-5-methyl-3-β-D-glucopyranos-1-yloxy-1H-pyrazole, at a dose of 10 mg/kg administered twice daily in the morning and evening, or with metformin at a dose of 200 mg/kg once daily in the morning, or with the combination of the pyrazole-O-glucoside and metformin at the same dose regimens. Vehicle for administration is 0.5% aqueous hydroxyethylcellulose containing 0.015% Polysorbat 80. Control animals are dosed twice daily with vehicle alone. Blood samples are obtained by tail bleed. Blood glucose is measured with a glucometer and HbA1c is measured with an automated analyzer (Cobas Integra 400, Roche). Prior to start of treatment, the animals are randomized for blood glucose and HbA1c (n=10/group).

On day 20 of treatment, blood glucose is measured in tail blood pre-dose, 0.5 h, 1.5 h, 3 h, and 7 h after dosing. Blood glucose is quantified by calculating the total glucose AUC 0-7 h. The data are presented as mean±SEM. The two-sided unpaired Student t-test is used for statistical comparison of the control group and the active groups.

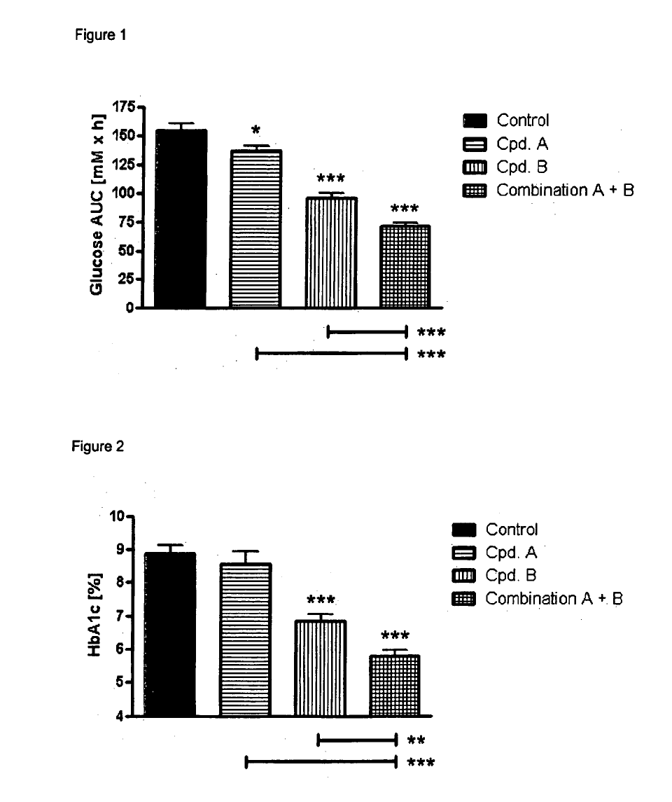

The result is shown in the following. FIG. 1. Cpd. A is metformin at a dose of 200 mg/kg once daily. Cpd. B is the pyrazole-O-glucoside (16) at a dose of 10 mg/kg administered twice daily. Combination A+B is the combination of the pyrazole-O-glucoside and metformin at the same dose regimens. P-values versus control are indicated by symbols above the bars. P-values of the combination versus the monotherapies are indicated below the figure (*, p<0.05; ***, p<0.001). Metformin reduces glucose AUC by 11%, the pyrazole-O-glucoside reduces glucose AUC by 38%. The combination decreases glucose AUC by 54%, and this reduction in glucose AUC is statistically significant versus each monotherapy.

On day 37 of treatment, HbA1c is measured pre-dose in tail blood obtained from overnight fasted animals. The data are presented as mean±SEM. The two-sided unpaired Student t-test is used for statistical comparison of the control group and the active groups.

The result is shown in the following FIG. 2. Cpd. A is metformin at a dose of 200 mg/kg once daily. Cpd. B is the pyrazole-O-glucoside (16) at a dose of 10 mg/kg administered twice daily. Combination A+B is the combination of the pyrazole-O-glucoside and metformin at the same dose regimens. P-values versus control are indicated by symbols above the bars. P-values of the combination versus the monotherapies are indicated below the figure (**, p<0.01; ***, p<0.001). Metformin reduces HbA1c numerically by 0.3%, the pyrazole-O-glucoside reduces HbA1c numerically by 2.1%.

The combination decreases HbA1c numerically by 3.1%, and this reduction in HbA1c is statistically significant versus each monotherapy.

### Examples for the Manufacture of pyrazole-O-glucosides

The following abbreviations are used above and hereinafter:

Bn benzyl

Bu butyl

DCM dichloromethane

DMF dimethylformamide

Et ethyl

EtOAc ethyl acetate

iPr iso-propyl

i. vac. in vacuo

Me methyl

Ph phenyl

RT ambient temperature (approx. 20° C.)

THF tetrahydrofuran

Preparation of Starting Materials:

### Example I

### 2-Fluoro-4-hydroxy-benzaldehyde

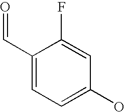

To a −70° C. solution of 2-fluoro-4-methoxy-benzaldehyde (19.1 g, 120 mmol) in CH₂Cl₂ (100 mL) was added boron tribromide in CH₂Cl₂ (1 M, 160 mL, 160 mmol). After stirring the reaction solution at −68° C. for 45 min the cooling bath was removed, and the solution was further stirred at room temperature over night. The reaction solution was poured into ice water and stirred for 30 min. The formed precipitate was separated, washed with CH₂Cl₂, and dissolved in EtOAc. The resultant EtOAc phase was washed with water and dried over MgSO₄. After evaporation of the solvent the residue was washed with little CH₂Cl₂ and dried in vacuo to give the product as a beige solid.

Yield: 14.5 g (86%)

ESI-MS: m/z=139 [M−H]⁻

### Example II

### 4-Benzyloxy-3-fluoro-benzaldehyde

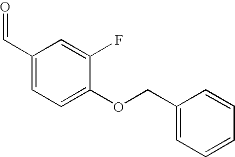

To a suspension of 4-hydroxy-3-fluoro-benzaldehyde (10.0 g, 70 mmol) and potassium carbonate (10.2 g, 74 mmol) in DMF (60 mL) was added dropwise benzyl bromide (8.7 mL, 74 mmol). The mixture was stirred at ambient temperature for 48 h and subsequently quenched with ice water. The mixture was further diluted with water, and the precipitate was separated by filtration. The precipitate was washed with water and dissolved in ethyl acetate. The organic solution was washed with brine, dried over sodium sulfate, and the solvent was removed in vacuo.

Yield: 16.0 g (99%)

ESI-MS: m/z=231 [M+H]⁺

In an analogous manner the following compounds can be obtained:

### (1) 4-Benzyloxy-2-fluoro-benzaldehyde

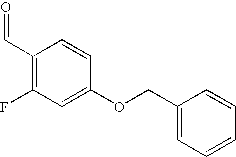

ESI-MS: m/z=253 [M+Na]⁺

### (2) 2-Chloro-4-methoxy-1-methyl-benzene

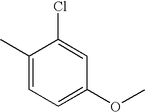

The procedure above was followed except for benzyl bromide methyl iodide was employed as the electrophile.

ESI-MS: m/z=156/158 [M]⁺ (chlorine)

### Example III

### 2,5-Difluoro-4-methoxy-benzaldehyde

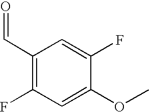

To a −65° C. solution of 1-bromo-2,5-difluoro-4-methoxy-benzene (25.0 g, 0.11 mol) in THF (150 mL) and Et₂O (250 mL) under Ar was added dropwise n-BuLi in hexane (1.6 M, 70 mL, 0.11 mol). After stirring the solution at −65° C. for 45 min, DMF (10 mL, 0.13 mol) was added slowly. The solution was warmed up in the cooling bath to room temperature over night and then diluted with Et₂O (500 mL). The resultant organic solution was washed with brine, dried over MgSO₄, and the solvent was removed in vacuo. The residue was recrystallized from iPr₂O to give the product as yellow crystals.

Yield: 6.7 g (35%)

R_{f} 0.63 (silica gel, petrol ether/EtOAc 1:1)

In an analogous manner the following compounds can be obtained:

### (1) 2,6-Difluoro-4-methoxy-benzaldehyde

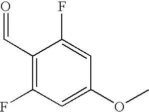

ESI-MS: m/z=173 [M+H]⁺

### (2) 3,5-Difluoro-4-methoxy-benzoic acid

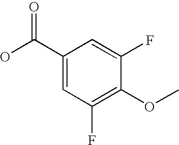

The procedure above was followed except for the quenching of the aryllithium compound with crushed dry ice (CO₂) instead of DMF.

ESI-MS: m/z=187 [M−H]⁻

### Example IV

### (4-Benzyloxy-3-fluoro-phenyl)-methanol

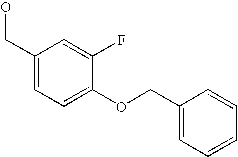

To a suspension of sodium borohydride (3.4 g, 90 mmol) in THF (60 mL) was added a solution of 4-benzyloxy-3-fluoro-benzaldehyde (16.1 g, 70 mmol) in THF (60 mL). After stirring at ambient temperature over night, the reaction mixture was quenched by the addition of ice water. The mixture was acidified with aqueous HCl (4 M) and extracted with Et₂O. The combined organic phases were washed with aqueous NaHCO₃ solution and dried over sodium sulfate. After removal of the solvent, the product was yielded.

Yield: 16.2 g (100%)

ESI-MS: m/z=215 [M-OH]⁺

In an analogous manner the following compounds can be prepared:

### (1) (2,5-Difluoro-4-methoxy-phenyl)-methanol

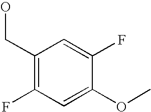

ESI-MS: m/z=215 [M-OH]⁺

### (2) (4-Benzyloxy-2-fluoro-phenyl)-methanol

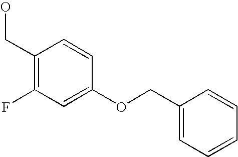

ESI-MS: m/z=232 [M]⁺

### (3) (2-Fluoro-4-methoxy-phenyl)-methanol

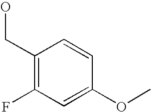

ESI-MS: m/z=139 [M-OH]⁺

### (4) (2,6-Difluoro-4-methoxy-phenyl)-methanol

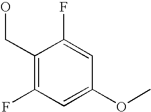

ESI-MS: m/z=157 [M-OH+H]⁺

### Example V

### (3,5-Difluoro-4-methoxy-phenyl)-methanol

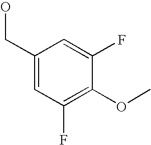

To a 20° C. suspension of lithium aluminumhydride (0.57 g, 15 mmol) in THF (50 mL) and toluene (30 mL) was added a solution of 3,5-difluoro-4-methoxy-benzoic acid (2.9 g, 15 mmol) in THF (20 mL). After stirring the reaction mixture at ambient temperature over night, ice water was added, and the resultant solution was acidified with 2 N sulfuric acid. The organic layer was separated and the aqueous extracted with EtOAc. The combined organic phases were washed with aqueous NaHCO₃ solution and brine and dried over MgSO₄. After removal of the solvent, the residue was purified by chromatography on silica gel (petrol ether/EtOAc 2:1).

Yield: 1.6 g (60%)

R_{f} 0.7 (silica gel, petrol ether/EtOAc 1:1)

### Example VI

### 1-Benzyloxy-4-bromomethyl-2-fluoro-benzene

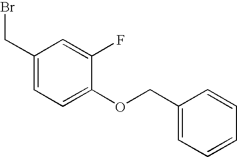

To an ice-cold solution of (4-benzyloxy-3-fluoro-phenyl)-methanol (16.7 g, 72 mmol) in diethylether (130 mL) was added phosphorous tribromide (2.8 mL, 30 mmol) at a rate such that the solution temperature did not exceed 8° C. After stirring at room temperature for 2 h, the reaction mixture was cooled in an ice-bath and quenched by the addition of ice water, ethyl acetate, and Et₂O. The organic layer was separated and washed with aqueous NaHCO₃ solution and brine. The product was yielded after evaporation of the solvent.

Yield: 20.5 g (97%)

ESI-MS: m/z=294/296 [M]⁺ (bromine)

In an analogous manner the following compounds were prepared:

### (1) 1-Bromomethyl-2,5-difluoro-4-methoxy-benzene

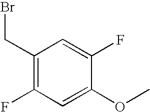

ESI-MS: m/z=236/238 [M]⁺ (bromine)

### (2) 4-Benzyloxy-1-bromomethyl-2-fluoro-benzene

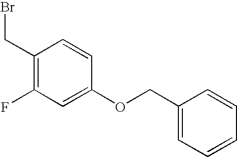

ESI-MS: m/z=294/296 [M]⁺ (bromine)

### (3) 1-Bromomethyl-2-fluoro-4-methoxy-benzene

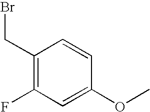

R_{f} 0.8 (silica gel, petrol ether/EtOAc 1:1)

### (4) 2-Bromomethyl-1,3-difluoro-5-methoxy-benzene

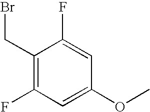

ESI-MS: m/z=236/238 [M]⁺ (bromine)

### (5) 5-Bromomethyl-1,3-difluoro-2-methoxy-benzene

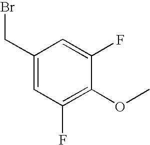

ESI-MS: m/z=236/238 [M]⁺ (bromine)

### Example VII

### 2,3-Difluoro-1-methoxy-4-methyl-benzene

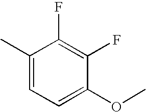

To a 20° C. solution of sodium hydroxide (14.4 g, 0.36 mol) and 2,3-difluoro-4-methyl-phenol (50.0 g, 0.35 mol) in water (160 mL) was added dropwise dimethyl sulfate (34 mL, 0.36 mol). After stirring at room temperature over night, the reaction solution was extracted with Et₂O. The ether phase was washed with 2 N NaOH solution, water, and brine and subsequently dried over MgSO₄. After removal of the solvent under reduced pressure, the product was yielded as a colorless oil.

Yield: 49.0 g (89%)

ESI-MS: m/z=158 [M]⁺

### Example VIII

### 1-Bromomethyl-2,3-difluoro-4-methoxy-benzene

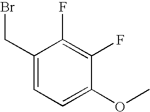

A solution of 2,3-difluoro-1-methoxy-4-methyl-benzene (39.5 g, 0.25 mol), N-bromo succinimide (44.5 g, 0.25 mol), and azobisisobutyronitrile (0.41 g, 2.5 mmol) in CCl₄ (300 mL) was stirred at reflux for 3.5 h. Then the formed succinimide was removed by filtration, and the filtrate was concentrated in vacuo. The residue was dissolved in Et₂O (200 mL) and concentrated to about 100 mL. After cooling in an ice-bath the formed precipitate was filtered off, washed with cold Et₂O, and dried in vacuo to give the product as a white solid.

Yield: 36.0 g (61%)

R_{f} 0.3 (silica gel, petrol ether/EtOAc 20:1)

The following compounds can be obtained by analogy with the procedure described above:

### (1) 4-Bromomethyl-2-chloro-1-methoxy-benzene

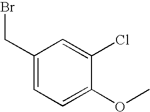

R_{f} 0.4 (silica gel, petrol ether/EtOAc 20:1)

### (2) 1-Bromomethyl-2-chloro-4-methoxy-benzene

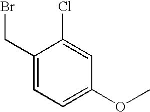

R_{f} 0.5 (silica gel, petrol ether/EtOAc 20:1)

### Example IX

### 2-(2,3-Difluoro-4-methoxy-benzyl)-3-oxo-butyric acid ethyl ester

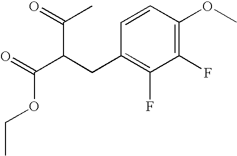

To an ice-cold suspension of sodium hydride (4.8 g, 120 mmol, 60% in mineral oil, freed from oil with pentane) in THF (140 mL) was added 3-oxo-butyric acid ethyl ester (17.2 g, 132 mmol) in THF (50 mL). After removing the ice-bath and stirring the solution at room temperature for 0.5 h, a solution of 1-methoxy-4-bromomethyl-2,3-difluoro-benzene (28.4 g, 120 mmol) in THF (60 mL) was added dropwise. After stirring the reaction mixture at reflux over night, the solvent was removed in vacuo and the residue was triturated with Et₂O (300 mL). The ether phase was washed with water and brine and dried over MgSO₄. The product was furnished as a yellow oil after evaporation of the solvent.

Yield: 35.5 g (ca. 80% pure)

ESI-MS: m/z=285 [M−H]⁻

The following compounds can be obtained in an analogous manner:

### (1) 2-(4-Benzyloxy-3-fluoro-benzyl)-3-oxo-butyric acid ethyl ester

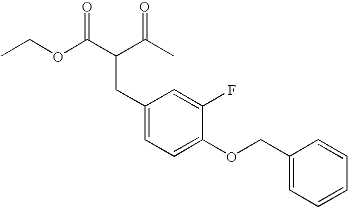

ESI-MS: m/z=345 [M+H]⁺

### (2) 2-(4-Iodo-benzyl)-3-oxo-butyric acid ethyl ester

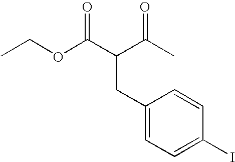

ESI-MS: m/z=345 [M−H]⁻

### (3) 2-(2,5-Difluoro-4-methoxy-benzyl)-3-oxo-butyric acid ethyl ester

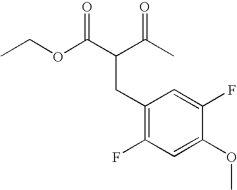

R_{f} 0.27 (silica gel, petrol ether/EtOAc 4:1)

### (4) 2-(4-Benzyloxy-2-fluoro-benzyl)-3-oxo-butyric acid ethyl ester

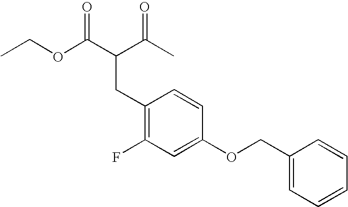

ESI-MS: m/z=343 [M−H]⁻

### (5) 2-(2,6-Difluoro-4-methoxy-benzyl)-3-oxo-butyric acid ethyl ester

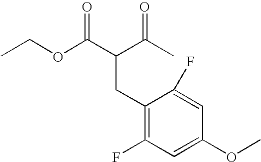

ESI-MS: m/z=287 [M+H]⁺

### (6) 2-(3,5-Difluoro-4-methoxy-benzyl)-3-oxo-butyric acid ethyl ester

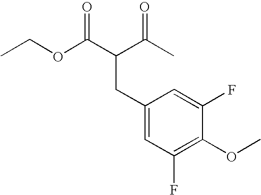

ESI-MS: m/z=287 [M+H]⁺

### (7) 2-(3-Fluoro-4-methyl-benzyl)-3-oxo-butyric acid ethyl ester

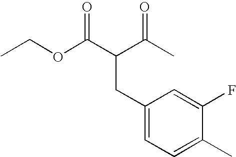

ESI-MS: m/z=253 [M+H]⁺

### (8) 2-(2-Fluoro-4-methoxy-benzyl)-3-oxo-butyric acid ethyl ester

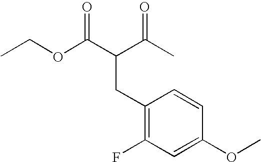

ESI-MS: m/z=269 [M+H]⁺

### (9) 2-(3-Chloro-4-methoxy-benzyl)-3-oxo-butyric acid ethyl ester

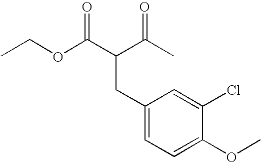

ESI-MS: m/z=283/285 [M−H]⁻ (chlorine)

### (10) 2-(2-Chloro-4-methoxy-benzyl)-3-oxo-butyric acid ethyl ester

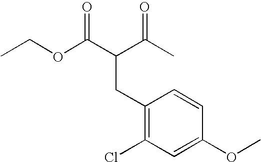

ESI-MS: m/z=285/287 [M+H]⁺ (chlorine)

### (11) 4,4,4-Trifluoro-2-(2-fluoro-4-methoxy-benzyl)-3-oxo-butyric acid ethyl ester

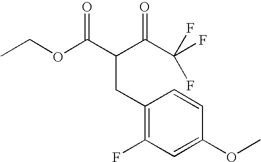

ESI-MS: m/z=321 [M−H]⁻

### Example X

### 2-(2,3-Difluoro-4-methyl-benzyl)-3-oxo-butyric acid ethyl ester

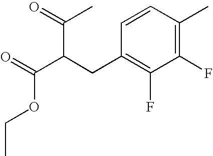

To an ice-cold solution of 3-oxo-butyric acid ethyl ester (4.17 g, 32.1 mmol) and sodium iodide (23.9 g, 160 mmol) under Ar in acetonitrile (220 mL) was added over 3 min trimethylsilyl chloride (20.2 mL, 160 mmol) followed by 2,3-difluoro-4-methyl-benzaldehyde (5.0 g, 32.1 mmol). The ice bath was removed, and the reaction mixture was stirred at room temperature for 8 h and subsequently at 60° C. for 15 h. After cooling to room temperature the reaction mixture was poured into a mixture of EtOAc (300 mL) and water (200 mL). The organic phase was separated and washed with aqueous Na₂S₂O₃ solution and brine and dried over Na₂SO₄. The solvent was removed under reduced pressure, and the residue was purified by silica gel chromatography (hexane/EtOAc 1:6) to give the product as a colorless oil.

Yield: 8.4 g (97%)

R_{f} 0.35 (silica gel, hexane/EtOAc 5:1)

The following compounds can be obtained in an analogous manner:

### (1) 2-(4-Bromo-3-fluoro-benzyl)-3-oxo-butyric acid ethyl ester

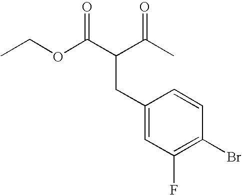

### (2) 2-(4-Bromo-2-fluoro-benzyl)-3-oxo-butyric acid ethyl ester

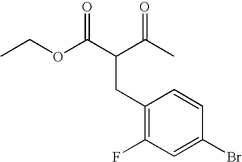

R_{f} 0.42 (silica gel, hexane/EtOAc 4:1)

### (3) 2-(2-fluoro-4-methyl-benzyl)-3-oxo-butyric acid ethyl ester

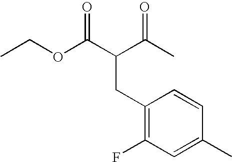

### Example XI

### 4,4,4-Trifluoro-2-(2-fluoro-4-methoxy-benzyl)-3-methoxy-but-2-enoic acid ethyl ester

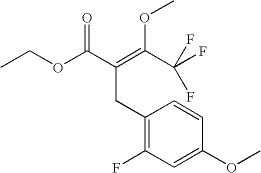

To a 20° C. mixture of 4,4,4-trifluoro-2-(2-fluoro-4-methoxy-benzyl)-3-oxo-butyric acid ethyl ester (6.35 g, 19.7 mmol) and cesium carbonate (9.5 g, 28.9 mmol) in DMF (50 mL) was dropped a solution of toluene-4-sulfonic acid methyl ester (4.5 g, 23.7 mmol) in DMF (20 mL). The reaction mixture was stirred at room temperature over night and subsequently at 60° C. for 1.5 h. After cooling to room temperature diluted phosphoric acid was added, and the resultant solution was extracted with Et₂O. The combined organic phases were washed with brine and dried over Na₂SO₄. After removal of the solvent the residue was purified by chromatography on aluminum oxide (cyclohexane/EtOAc 99:1→70:30).

Yield: 6.6 g (100%)

ESI-MS: m/z=337 [M+H]⁺

### Example XII

### 4-(2,3-Difluoro-4-methoxy-benzyl)-5-methyl-1H-pyrazol-3-ol

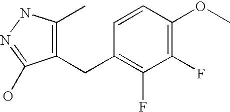

A solution of 2-(2,3-difluoro-4-methoxy-benzyl)-3-oxo-butyric acid ethyl ester (33.0 g, 0.115 mol) and hydrazine hydrate (80%, 8.0 g, 128 mmol) in EtOH (300 mL) was stirred at reflux for 2 h. After cooling in an ice-bath the precipitate was collected, washed with cold EtOH, and dried in vacuo to give the product as a white solid.

Yield: 22.5 g (70%)

ESI-MS: m/z=255 [M+H]⁺

The following compounds can be obtained accordingly:

### (1) 4-(4-Benzyloxy-3-fluoro-benzyl)-5-methyl-1H-pyrazol-3-ol

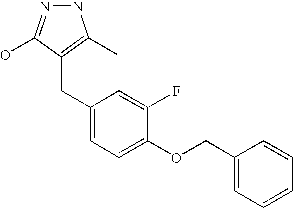

ESI-MS: m/z=313 [M+H]⁺

### (2) 4-(4-Iodo-benzyl)-5-methyl-1H-pyrazol-3-ol

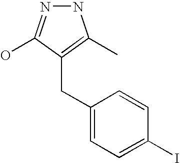

ESI-MS: m/z=315 [M+H]⁺

### (3) 4-(2,5-Difluoro-4-methoxy-benzyl)-5-methyl-1H-pyrazol-3-ol

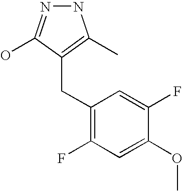

ESI-MS: m/z=255 [M+H]⁺

### (4) 4-(4-Benzyloxy-2-fluoro-benzyl)-5-methyl-1H-pyrazol-3-ol

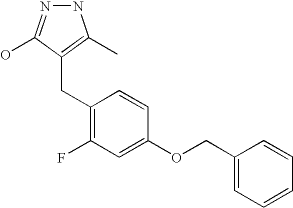

ESI-MS: m/z=313 [M+H]⁺

### (5) 4-(2,6-Difluoro-4-methoxy-benzyl)-5-methyl-1H-pyrazol-3-ol

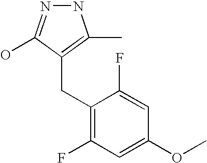

ESI-MS: m/z=255 [M+H]⁺

### (6) 4-(3,5-Difluoro-4-methoxy-benzyl)-5-methyl-1H-pyrazol-3-ol

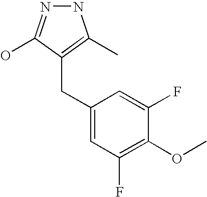

ESI-MS: m/z=255 [M+H]⁺

### (7) 4-(3-Fluoro-4-methyl-benzyl)-5-methyl-1H-pyrazol-3-ol

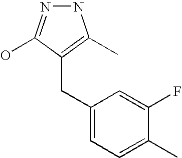

ESI-MS: m/z=221 [M+H]⁺

### (8) 4-(2-Fluoro-4-methoxy-benzyl)-5-methyl-1H-pyrazol-3-ol

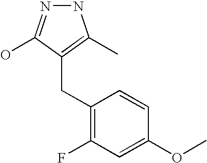

ESI-MS: m/z=237 [M+H]⁺

### (9) 4-(3-Chloro-4-methoxy-benzyl)-5-methyl-1H-pyrazol-3-ol

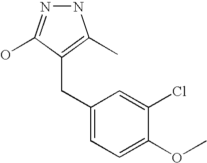

ESI-MS: m/z=253/255 [M+H]⁺ (chlorine)

### (10) 4-(2-Chloro-4-methoxy-benzyl)-5-methyl-1H-pyrazol-3-ol

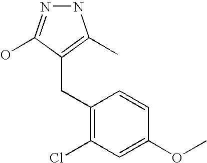

ESI-MS: m/z=253/255 [M+H]⁺ (chlorine)

### (11) 4-(2-Fluoro-4-methoxy-benzyl)-5-trifluoromethyl-1H-pyrazol-3-ol

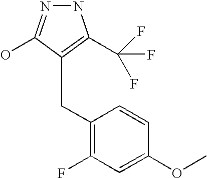

The product was prepared following the procedure above starting from 4,4,4-trifluoro-2-(2-fluoro-4-methoxy-benzyl)-3-methoxy-but-2-enoic acid ethyl ester

ESI-MS: m/z=289 [M−H]⁻

### (12) 4-(4-Bromo-3-fluoro-benzyl)-5-methyl-1H-pyrazol-3-ol

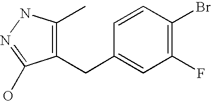

### (13) 4-(2,3-Difluoro-4-methyl-benzyl)-5-methyl-1H-pyrazol-3-ol

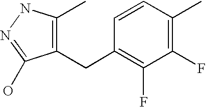

R_{f} 0.05 (silica gel, hexane/EtOAc 5:1)

### (14) 4-(4-Bromo-2-fluoro-benzyl)-5-methyl-1H-pyrazol-3-ol

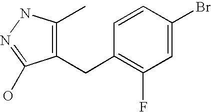

R_{f} 0.15 (silica gel, hexane/EtOAc 1:1)

### (15) 4-(2-Fluoro-4-methyl-benzyl)-5-methyl-1H-pyrazol-3-ol

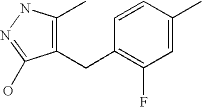

R_{f} 0.11 (silica gel, hexane/EtOAc 1:1)

### Example XIII

### 3-(tert-Butyl-dimethyksilyloxy)-4-(2-fluoro-4-methoxy-benzyl)-5-trifluoromethyl-1H-pyrazole

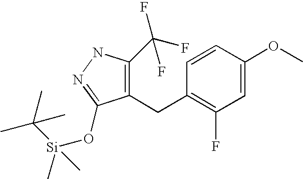

To a solution of 4-(2-fluoro-4-methoxy-benzyl)-5-trifluoromethyl-1H-pyrazol-3-ol (0.21 g, 0.72 mmol) and imidazole (8.0 g, 128 mmol) in DMF (2 mL) was added tert-butyldimethylsilylchloride (0.13 g, 0.86 mmol). After stirring at room temperature for 4 h, the solution was diluted with EtOAc and washed with water and brine. The organic phase was dried and the solvent removed.

Yield: 0.34 g (ca. 80% pure)

ESI-MS: m/z=405 [M+H]⁺

### Example XIV

### 3-(tert-Butyl-dimethyl-silyloxy)-4-(2-fluoro-4-methoxy-benzyl)-1-isopropyl-5-trifluoromethyl-1H-pyrazole

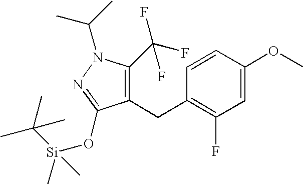

To a suspension of 3-(tert-butyl-dimethyl-silyloxy)-4-(2-fluoro-4-methoxy-benzyl)-5-trifluoromethyl-1H-pyrazole (0.27 g, 0.67 mmol) and Ph₃P (0.20 g, 0.76 mmol) in isopropanol (2 mL) was added diethyl azodicarboxylate in toluene (40%, 0.35 mL, 0.76 mmol). The solution was stirred at room temperature for 1 h and then diluted with Et₂O. The resultant solution was washed with water and aqueous NaOH solution (2 N), dried over Na₂SO₄, and the solvent was removed. The residue was purified by chromatography on silica gel (cyclohexane/EtOAc 99:1→4:1) to give the product as a colorless oil.

Yield: 0.14 g (47%)

ESI-MS: m/z=447 [M+H]⁺

### Example XV

### 4-(2-Fluoro-4-methoxy-benzyl)-1-isopropyl-5-trifluoromethyl-1H-pyrazol-3-ol

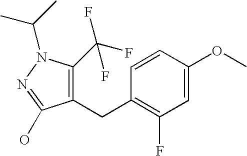

A solution of 3-(tert-butyl-dimethyl-silyloxy)-4-(2-fluoro-4-methoxy-benzyl)-1-isopropyl-5-trifluoromethyl-1H-pyrazole (0.27 g, 0.67 mmol), aqueous HCl (1 N, 1 mL, 1 mmol), MeOH (0.5 mL), and THF (12 mL) was stirred at 60° C. for 2 h. After cooling to room temperature the solution was diluted with EtOAc and washed with water and brine. The product was yielded as a white solid after drying over Na₂SO₄ and removal of the solvent in vacuo.

Yield: 0.10 g (100%)

ESI-MS: m/z=333 [M+H]⁺

### Example XVI

### 4-(2,3-Difluoro-4-methoxy-benzyl)-5-methyl-3-(2,3,4,6-tetra-O-benzyl-β-D-glycopyranos-1-yloxy)-1H-pyrazole

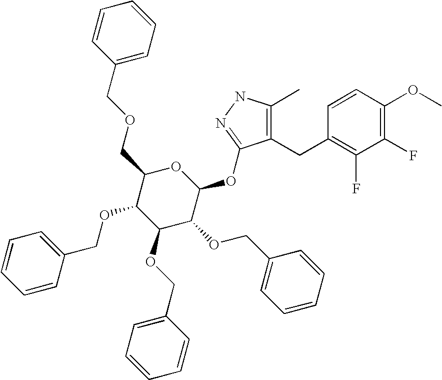

To a 0° C. solution of 4-(2,3-Difluoro-4-methoxy-benzyl)-5-methyl-1H-pyrazol-3-ol (2.14 g, 8.4 mmol), 2,3,4,6-tetra-O-benzyl-α-D-gluco-pyranose (4.54 g, 8.4 mmol), and PPh₃ (2.20 g, 8.4 mmol) in dry THF (80 mL) was added diethyl azodicarboxylate in toluene (40%, 3.85 mL, 8.4 mmol) at a rate such that the solution maintained 2-6° C. After 10 min the cooling bath was removed, and the reaction solution was stirred at room temperature over night. Then the solution was concentrated at 40° C. under reduced pressure, and the remainder was treated with Et₂O (50 mL). The ether solution was cooled to −18° C., and the forming precipitate was separated and washed with cold Et₂O. The filtrate was diluted with Et₂O and washed with aqueous NaOH solution (2 N), water, and brine. After drying over MgSO₄ and evaporation of the solvent, the residue was purified by chromatography on silica gel (cyclohexane/EtOAc 2:1→1:6). The purified product was recrystallized from iPr₂O to give the product as a white solid (<5% α anomer).

Yield: 3.10 g (48%)

ESI-MS: m/z=777 [M+H]⁺

The following compounds can be obtained accordingly:

### (1) 4-(2,5-Difluoro-4-methoxy-benzyl)-5-methyl-3-(2,3,4,6-tetra-O-benzyl-β-D-glucopyranos-1-yloxy)-1H-pyrazole

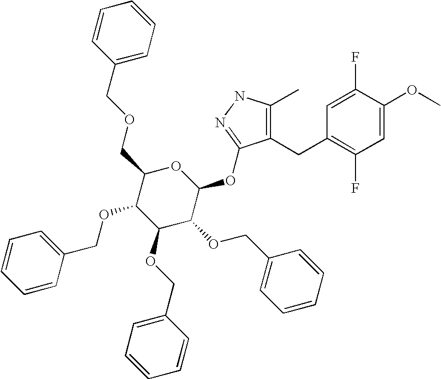

ESI-MS: m/z=777 [M+H]⁺

### (2) 4-(2-Fluoro-4-benzyloxy-benzyl)-5-methyl-3-(2,3,4,6-tetra-O-benzyl-β-D-glucopyranos-1-yloxy)-1H-pyrazole

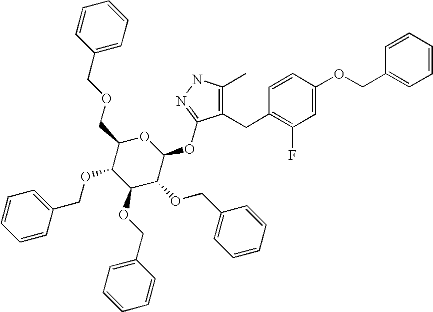

ESI-MS: m/z=835 [M+H]⁺

### (3) 4-(2,6-Difluoro-4-methoxy-benzyl)-5-methyl-3-(2,3,4,6-tetra-O-benzyl-β-D-glucopyranos-1-yloxy)-1H-pyrazole

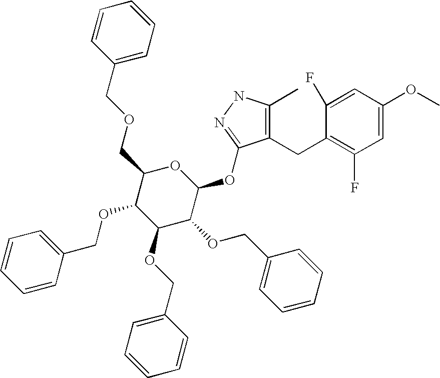

ESI-MS: m/z=777 [M+H]⁺

### (4) 4-(3,5-Difluoro-4-methoxy-benzyl)-5-methyl-3-(2,3,4,6-tetra-O-benzyl-β-D-glucopyranos-1-yloxy)-1H-pyrazole

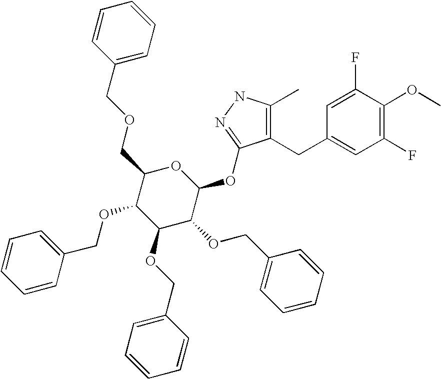

ESI-MS: m/z=777 [M+H]⁺

### (5) 4-(3-Fluoro-4-methyl-benzyl)-5-methyl-3-(2,3,4,6-tetra-O-benzyl-β-D-glucopyranos-1-yloxy)-1H-pyrazole

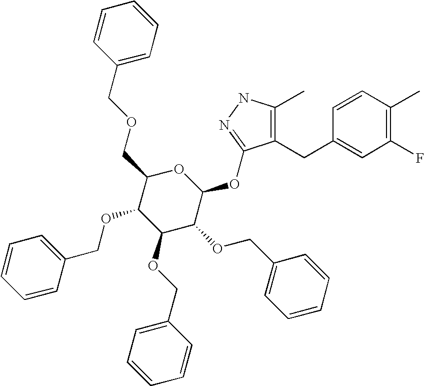

Bu₃P and 1,1′-(azodicarbonyl)-dipiperidine were used instead of Ph₃P and diethyl azodicarboxylate

ESI-MS: m/z=743 [M+H]⁺

### (6) 4-(2-Fluoro-4-methoxy-benzyl)-5-methyl-3-(2,3,4,6-tetra-O-benzyl-β-D-glucopyranos-1-yloxy)-1H-pyrazole

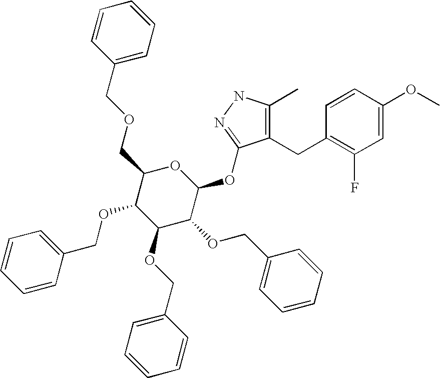

Bu₃P and 1,1′-(azodicarbonyl)-dipiperidine were used instead of Ph₃P and diethyl azodicarboxylate

ESI-MS: m/z=759 [M+H]⁺

### (7) 4-(3-Chloro-4-methoxy-benzyl)-5-methyl-3-(2,3,4,6-tetra-O-benzyl-β-D-glucopyranos-1-yloxy)-1H-pyrazole

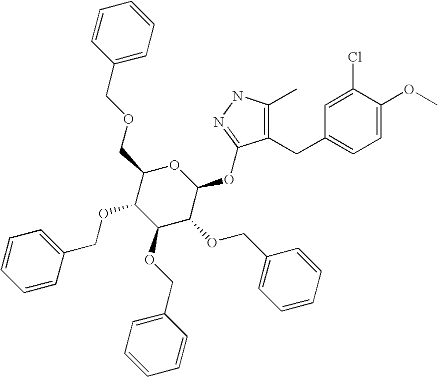

Bu₃P and 1,1′-(azodicarbonyl)-dipiperidine were used instead of Ph₃P and diethyl azodicarboxylate

ESI-MS: m/z=775/777 [M+H]⁺ (chlorine)

### (8) 4-(2-Chloro-4-methoxy-benzyl)-5-methyl-3-(2,3,4,6-tetra-O-benzyl-β-D-glucopyranos-1-yloxy)-1H-pyrazole

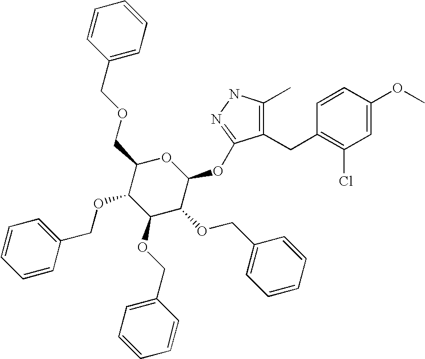

Bu₃P and 1,1′-(azodicarbonyl)-dipiperidine were used instead of Ph₃P and diethyl azodicarboxylate

ESI-MS: m/z=775/777 [M+H]⁺ (chlorine)

### (9) 4-(4-Bromo-3-fluoro-benzyl)-5-methyl-3-(2,3,4,6-tetra-O-benzyl-β-D-glucopyranos-1-yloxy)-1H-pyrazole

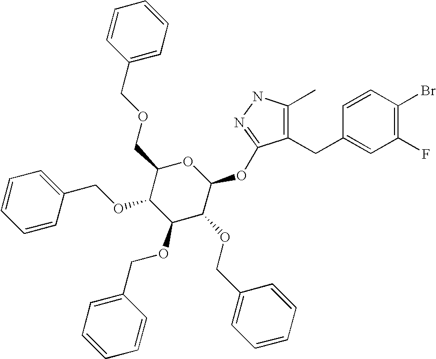

### (10) 4-(2,3-Difluoro-4-methyl-benzyl)-5-methyl-3-(2,3,4,6-tetra-O-benzyl-β-D-glucopyranos-1-yloxy)-1H-pyrazole

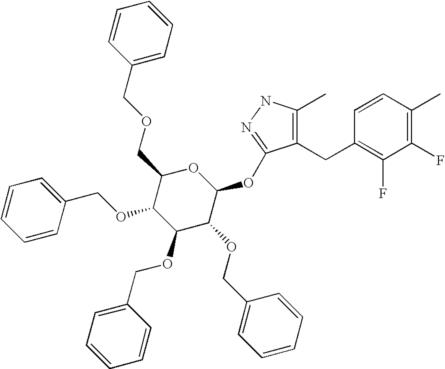

R_{f} 0.24 (silica gel, hexane/EtOAc 1:1)

### (11) 4-(2-Fluoro-4-methyl-benzyl)-5-methyl-3-(2,3,4,6-tetra-O-benzyl-β-D-glucopyranos-1-yloxy)-1H-pyrazole

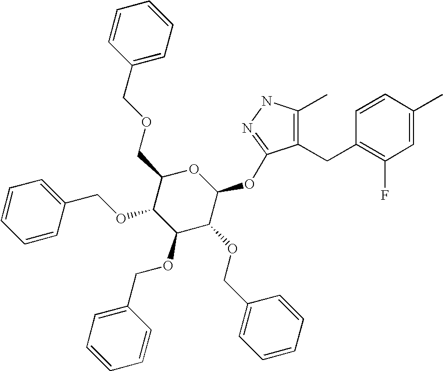

R_{f} 0.48 (silica gel, hexane/EtOAc 1:1)

### Example XVII

### 4-(4-iodo-benzyl)-5-methyl-3-(2,3,4,6-tetra-O-acetyl-β-D-glycopyranos-1-yloxy)-1H-pyrazole

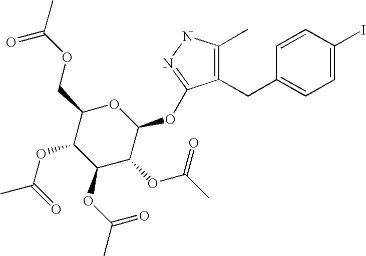

To a solution of 4-(4-iodo-benzyl)-5-methyl-1H-pyrazol-3-ol (0.70 g, 2.23 mmol) in dry THF (80 mL) was added Ag₂CO₃ (0.65 g, 2.36 mmol) followed by 2,3,4,6-tetra-O-acetyl-β-D-glucopyranos-1-ylbromide (1.00 g, 2.43 mmol). The reaction mixture was stirred at reflux in the dark over night prior to the addition of another portion of Ag₂CO₃ (0.75 g, 2.72 mmol) and 2,3,4,6-tetra-O-acetyl-β-D-glucopyranos-1-ylbromide (1.10 g, 2.68 mmol). The reaction mixture was stirred at reflux for another night and then cooled to room temperature. The mixture was filtrated, and the filtrate was concentrated in vacuo. The residue was purified by chromatography on silica gel (CH₂Cl₂/MeOH 1:0→10:1) to give the product as a white solid.

Yield: 0.40 g (28%)

ESI-MS: m/z=645 [M+H]⁺

The following compounds can be obtained accordingly:

### (1) 4-(4-benzyloxy-3-fluoro-benzyl)-5-methyl-3-(2,3,4,6-tetra-O-acetyl-β-D-glucopyranos-1-yloxy)-1H-pyrazole

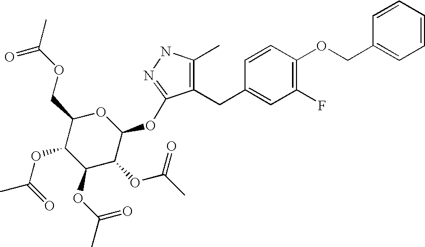

ESI-MS: m/z=643 [M+H]⁺

### (2) 4-(4-Bromo-2-fluoro-benzyl)-5-methyl-3-(2,3,4,6-tetra-O-acetyl-β-D-glucopyranos-1-yloxy)-1H-pyrazole

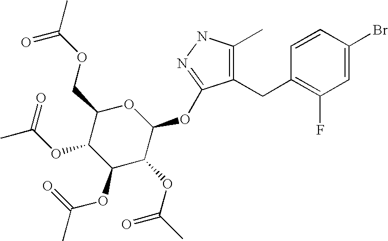

R_{f} 0.46 (silica gel, hexane/EtOAc 1:1)

### Example XVIII

### 4-(2-fluoro-4-methoxy-benzyl)-5-trifluoromethyl-3-(2,3,4,6-tetra-O-acetyl-β-D-glycopyranos-1-yloxy)-1H-pyrazole

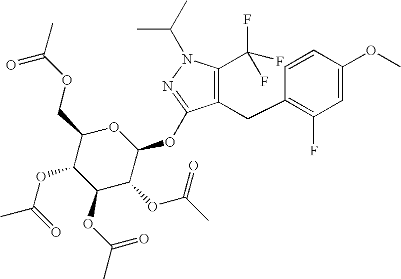

To a solution of 4-(2-Fluoro-4-methoxy-benzyl)-1-isopropyl-5-trifluoromethyl-1H-pyrazol-3-ol (1.84 g, 5.54 mmol), K₂CO₃ (7.5 g, 54.3 mmol), and nBu₃BnNCl (0.25 g, 0.8 mmol) in water (5 mL) and CH₂Cl₂ (25 mL) was added 2,3,4,6-tetra-O-acetyl-β-D-glucopyranos-1-ylbromide (3.80 g, 8.78 mmol). The reaction mixture was stirred vigorously at room temperature in the dark over night. Then CH₂Cl₂ was added and the organic layer was separated. After washing with water and 1 M phosphoric acid, the organic phase was dried over Na₂SO₄, and the solvent was removed. The residue was purified by chromatography on silica gel (cyclohexane/EtOAc 3:2→0:1).

Yield: 2.42 g (ca. 50% pure)

ESI-MS: m/z=663 [M+H]⁺

### Example XIX

### 4-(2,3-Difluoro-4-methoxy-benzyl)-1-isopropyl-5-methyl-3-(2,3,4,6-tetra-O-benzyl-β-D-glucopyranos-1-yloxy)-1H-pyrazole

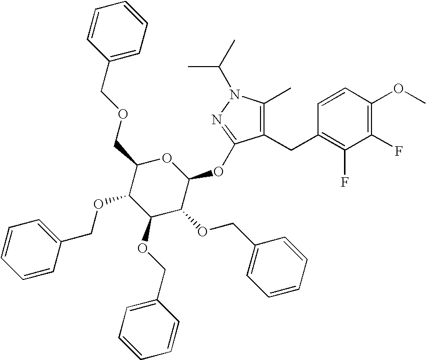

To a 20° C. mixture of 4-(2,3-difluoro-4-methoxy-benzyl)-5-methyl-3-(2,3,4,6-tetra-O-benzyl-β-D-glucopyranos-1-yloxy)-1H-pyrazole (2.90 g, 3.7 mmol) and Cs₂CO₃ (12.30 g, 37.8 mmol) in DMF (56 mL) was added isopropyl iodide (1.90 mL, 18.9 mmol). The reaction mixture was stirred at room temperature for 2.5 h. Then the reaction mixture was poured into water (300 mL), and the resultant solution was extracted with EtOAc. The combined organic extracts were washed with water and brine and dried over MgSO₄. The organic solution was concentrated at 40° C. under reduced pressure, and the residue was purified by chromatography on silica gel (cyclohexane/EtOAc 6:1→1:1).

Yield: 2.10 g (69%)

ESI-MS: m/z=459 [M+H]⁺

The following compounds can be obtained accordingly:

### (1) 4-(4-benzyloxy-3-fluoro-benzyl)-1-isopropyl-5-methyl-3-(2,3,4,6-tetra-O-acetyl-β-D-glucopyranos-1-yloxy)-1H-pyrazole

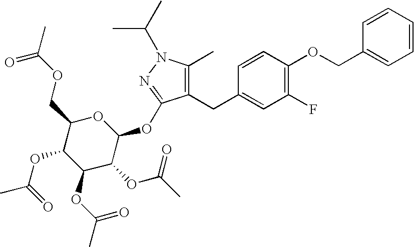

ESI-MS: m/z=685 [M+H]⁺

### (2) 4-(4-iodo-benzyl)-1-isopropyl-5-methyl-3-(2,3,4,6-tetra-O-acetyl-β-D-glucopyranos-1-yloxy)-1H-pyrazole

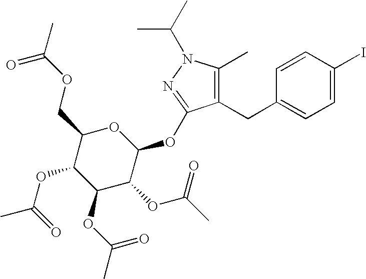

ESI-MS: m/z=687 [M+H]⁺

### (3) 4-(2,5-Difluoro-4-methoxy-benzyl)-1-isopropyl-5-methyl-3-(2,3,4,6-tetra-O-benzyl-β-D-glucopyranos-1-yloxy)-1H-pyrazole

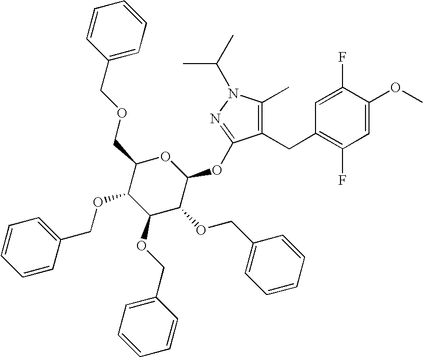

ESI-MS: m/z=819 [M+H]⁺

### (4) 4-(2-Fluoro-4-benzyloxy-benzyl)-1-isopropyl-5-methyl-3-(2,3,4,6-tetra-O-benzyl-β-D-glucopyranos-1-yloxy)-1H-pyrazole

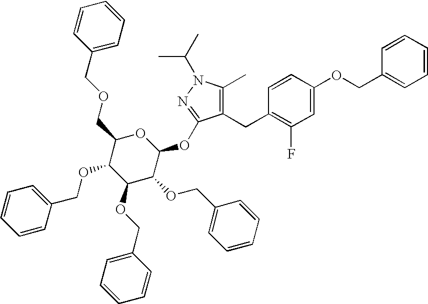

ESI-MS: m/z=877 [M+H]⁺

### (5) 4-(2,6-Difluoro-4-methoxy-benzyl)-1-isopropyl-5-methyl-3-(2,3,4,6-tetra-O-benzyl-β-D-glucopyranos-1-yloxy)-1H-pyrazole

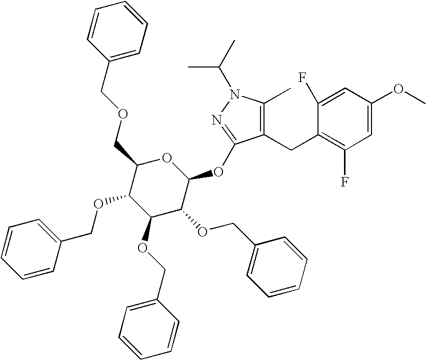

ESI-MS: m/z=819 [M+H]⁺

### (6) 4-(3,5-Difluoro-4-methoxy-benzyl)-1-isopropyl-5-methyl-3-(2,3,4,6-tetra-O-benzyl-β-D-glucopyranos-1-yloxy)-1H-pyrazole

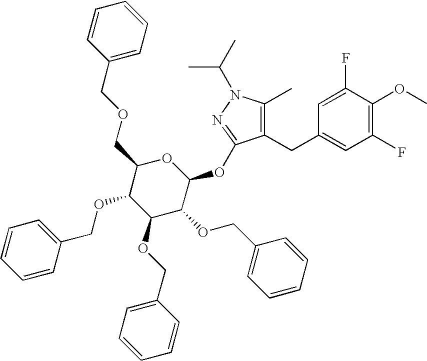

ESI-MS: m/z=819 [M+H]⁺

### (7) 1-Cyclobutyl-4-(3-fluoro-4-methyl-benzyl)-5-methyl-3-(2,3,4,6-tetra-O-benzyl-β-D-glucopyranos-1-yloxy)-1H-pyrazole

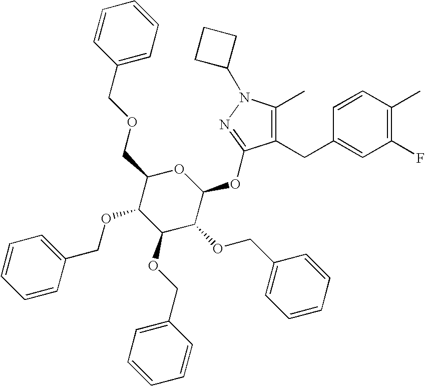

Bromo-cyclobutane was used as the electrophile instead of isopropyl iodide

ESI-MS: m/z=797 [M+H]⁺

### (8) 1-Cyclopropylmethyl-4-(3-fluoro-4-methyl-benzyl)-5-methyl-3-(2,3,4,6-tetra-O-benzyl-β-D-glucopyranos-1-yloxy)-1H-pyrazole

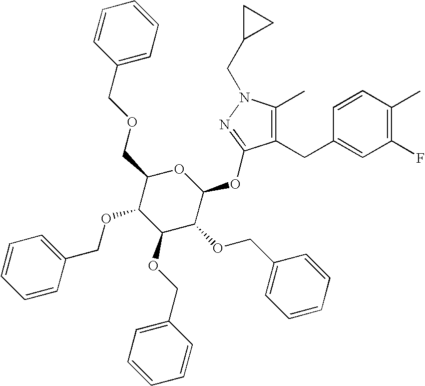

Yielded as a side product in the preparation of example XVIII (7)

ESI-MS: m/z=797 [M+H]⁺

### (9) 1-Cyclobutyl-4-(2-fluoro-4-methoxy-benzyl)-5-methyl-3-(2,3,4,6-tetra-O-benzyl-β-D-glucopyranos-1-yloxy)-1H-pyrazole

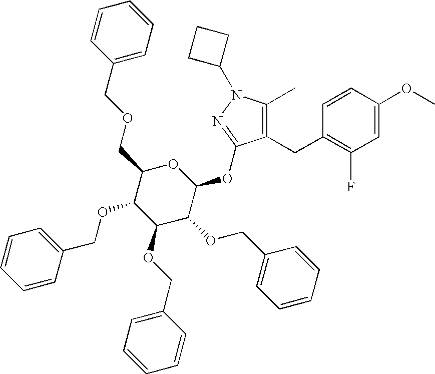

Bromo-cyclobutane was used as the electrophile instead of isopropyl iodide

ESI-MS: m/z=813 [M+H]⁺

### (10) 4-(3-Chloro-4-methoxy-benzyl)-1-isopropyl-5-methyl-3-(2,3,4,6-tetra-O-benzyl-β-D-glucopyranos-1-yloxy)-1H-pyrazole

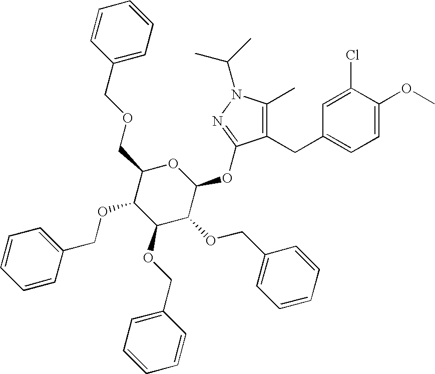

The reaction is preferably carried out with potassium hexamethyldisilazide as the base in toluene and THF

ESI-MS: m/z=817/819 [M+H]⁺ (chlorine)

### (11) 4-(2-Chloro-4-methoxy-benzyl)-1-isopropyl-5-methyl-3-(2,3,4,6-tetra-O-benzyl-β-D-glucopyranos-1-yloxy)-1H-pyrazole

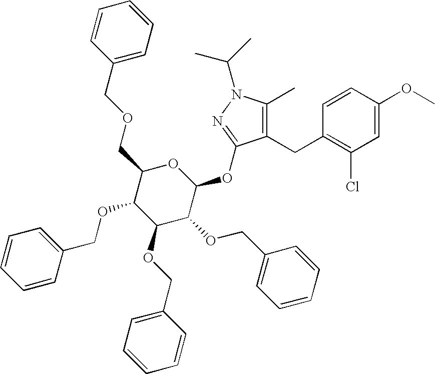

The reaction is preferably carried out with potassium hexamethyldisilazide as the base in toluene and THF

ESI-MS: m/z=817/819 [M+H]⁺ (chlorine)

### (12) 4-(4-Bromo-3-fluoro-benzyl)-1-isopropyl-5-methyl-3-(2,3,4,6-tetra-O-benzyl-β-D-glucopyranos-1-yloxy)-1H-pyrazole

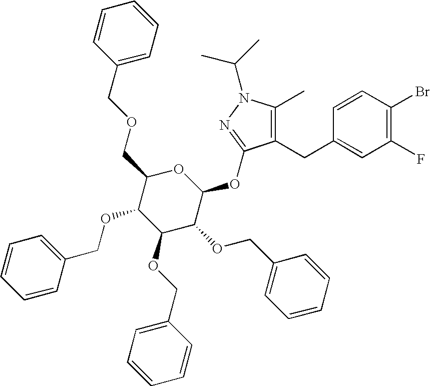

### (13) 4-(2,3-Difluoro-4-methyl-benzyl)-1-isopropyl-5-methyl-3-(2,3,4,6-tetra-O-benzyl-β-D-glucopyranos-1-yloxy)-1H-pyrazole

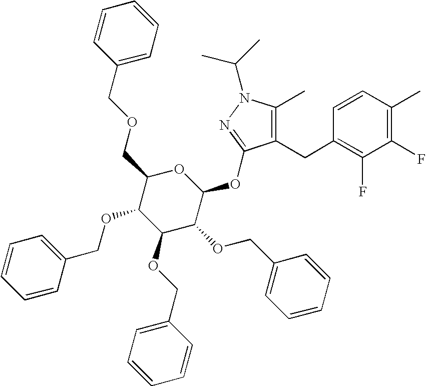

R_{f} 0.65 (silica gel, hexane/EtOAc 1:1)

### (14) 4-(4-Bromo-2-fluoro-benzyl)-1-isopropyl-5-methyl-3-(2,3,4,6-tetra-O-acetyl-β-D-glucopyranos-1-yloxy)-1H-pyrazole

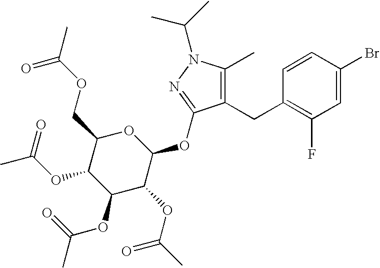

R_{f} 0.50 (silica gel, hexane/EtOAc 1:1)

### (15) 4-(2-Fluoro-4-methyl-benzyl)-1-isopropyl-5-methyl-3-(2,3,4,6-tetra-O-benzyl-β-D-glucopyranos-1-yloxy)-1H-pyrazole

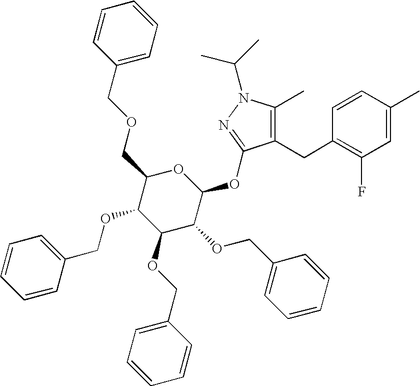

R_{f} 0.53 (silica gel, hexane/EtOAc 4:1)

### Example XX

### 4-(3-fluoro-4-hydroxy-benzyl)-1-isopropyl-5-methyl-3-(2,3,4,6-tetra-O-acetyl-β-D-glucopyranos-1-yloxy)-1H-pyrazole

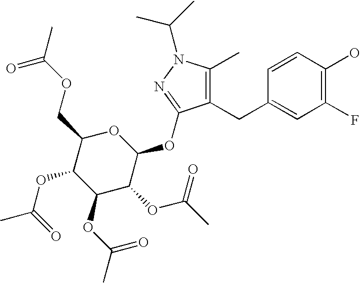

A mixture of 4-(4-benzyloxy-3-fluoro-benzyl)-1-isopropyl-5-methyl-3-(2,3,4,6-tetra-O-acetyl-β-D-glucopyranos-1-yloxy)-1H-pyrazole (0.26 g, 0.38 mmol) and 10% Pd on carbon (0.05 g) in EtOAc (10 mL) was stirred at room temperature under hydrogen atmosphere (3 bar). After 3 h the catalyst was separated by filtration, and the solvent was removed under reduced pressure. The residue was dissolved in Et₂O, filtered over Celite®, and concentrated in vacuo.

Yield: 0.22 g (97%)

ESI-MS: m/z=595 [M+H]⁺

### Example XXI

### 4-(3-Fluoro-4-ethoxy-benzyl)-1-isopropyl-5-methyl-3-(2,3,4,6-tetra-O-acetyl-β-D-glucopyranos-1-yloxy)-1H-pyrazole

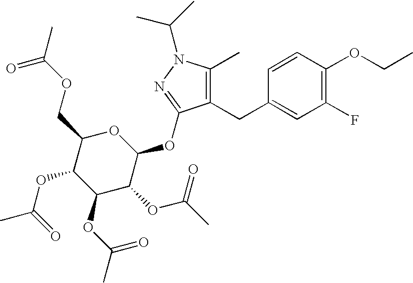

To a suspension of 4-(3-fluoro-4-hydroxy-benzyl)-1-isopropyl-5-methyl-3-(2,3,4,6-tetra-O-acetyl-β-D-glucopyranos-1-yloxy)-1H-pyrazole (0.22 g, 0.37 mmol) and cesium carbonate (0.31 g, 0.40 mmol) in DMF (3 mL) was added ethyl bromide (30 μL, 0.40 mmol). After stirring at ambient temperature for 5 h, the mixture was poured into a mixture of EtOAc and phosphoric acid (0.1 M). The organic phase was separated, washed with aqueous NaHCO₃ solution and brine, and dried over Na₂SO₄. The organic solution was concentrated, and the residue was purified by silica gel chromatography (petrol ether/EtOAc 1:1).

Yield: 0.18 g (78%)

ESI-MS: m/z=623 [M+H]⁺

The following compound can be obtained accordingly:

### (1) 4-(3-Fluoro-4-isopropoxy-benzyl)-1-isopropyl-5-methyl-3-(2,3,4,6-tetra-O-acetyl-β-D-glucopyranos-1-yloxy)-1H-pyrazole

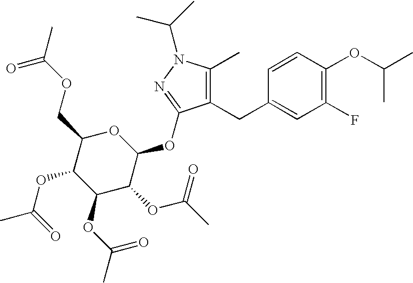

ESI-MS: m/z=637 [M+H]⁺

### Example XXII

### 4-(2-fluoro-4-hydroxy-benzyl)-1-isopropyl-5-methyl-3-β-D-glucopyranos-1-yloxy-1H-pyrazole

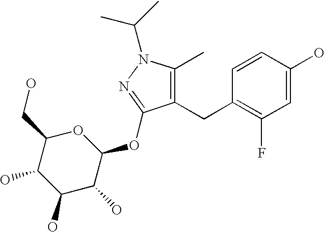

A mixture of 4-(2-fluoro-4-benzyloxy-benzyl)-1-isopropyl-5-methyl-3-(2,3,4,6-tetra-O-benzyl-β-D-glucopyranos-1-yloxy)-1H-pyrazole (2.0 g, 2.3 mmol) and 20% Pd on carbon (1.0 g) in EtOH (70 mL) was stirred at room temperature under hydrogen atmosphere (50 psi). After 2 h the catalyst was separated by filtration, and the solvent was removed under reduced pressure. The residue was purified by silica gel chromatography (CH₂Cl₂/MeOH 10:1→3:1).

Yield: 0.69 g (71%)

ESI-MS: m/z=427 [M+H]⁺

### Example XXIII

### 4-(4-Trimethylsilylethinyl-benzyl)-1-isopropyl-5-methyl-3-(2,3,4,6-tetra-O-acetyl-β-D-glucopyranos-1-yloxy)-1H-pyrazole

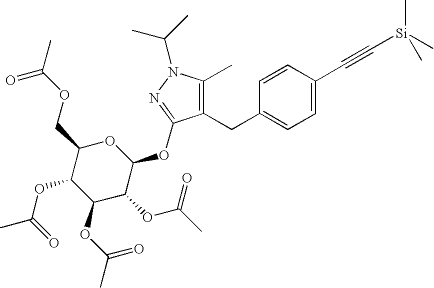

To a degassed solution of 4-(4-iodo-benzyl)-1-isopropyl-5-methyl-3-(2,3,4,6-tetra-O-acetyl-β-D-glucopyranos-1-yloxy)-1H-pyrazole (0.31 g, 0.45 mmol) in DMF (5 mL) under Ar was added in the given order NEt₃ (0.2 mL, 1.43 mmol), CuI (0.02 g, 0.11 mmol), (Ph₃P)₂PdCl₂ (0.05 g, 0.07 mmol), and trimethylsilylacetylen (0.10 mL, 0.69 mmol). The reaction mixture was stirred at 90° C. for 3.5 h. After cooling to room temperature EtOAc was added, and the resultant solution was washed with aqueous NaHCO₃ solution and dried over Na₂SO₄. The solvent was evaporated, and the residue was purified by chromatography on silica gel (cyclohexane/EtOAc 9:1→1:1) to give the product as a yellow oil.

Yield: 0.18 g (64%)

ESI-MS: m/z=657 [M+H]⁺

Preparation of Products

### Example 1

### (1) 4-(2,3-Difluoro-4-methoxy-benzyl)-1-isopropyl-5-methyl-3-β-D-glucopyranos-1-yloxy-1H-pyrazole

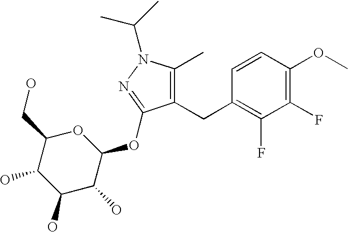

A mixture of 4-(2,3-difluoro-4-methoxy-benzyl)-1-isopropyl-5-methyl-3-(2,3,4,6-tetra-O-benzyl-β-D-glucopyranos-1-yloxy)-1H-pyrazole (1.80 g, 2.2 mmol) and 20% Pd on carbon (1 g) in EtOH (50 mL) was stirred at room temperature under hydrogen atmosphere (50 psi). After 2.5 h the catalyst was separated by filtration, and the solvent was removed under reduced pressure. The residue was purified by chromatography on silica gel (DCM/MeOH 1:0→4:1) to afford the product as a white solid.

Yield: 0.48 g (48%)

ESI-MS: m/z=459 [M+H]⁺

The following compounds can be obtained accordingly:

### (2) 4-(2,5-Difluoro-4-methoxy-benzyl)-1-isopropyl-5-methyl-3-β-D-glucopyranos-1-yloxy-1H-pyrazole

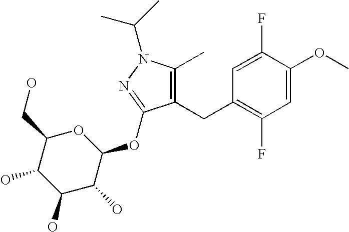

ESI-MS: m/z=459 [M+H]⁺

### (3) 4-(2,6-Difluoro-4-methoxy-benzyl)-1-isopropyl-5-methyl-3-β-D-glucopyranos-1-yloxy-1H-pyrazole

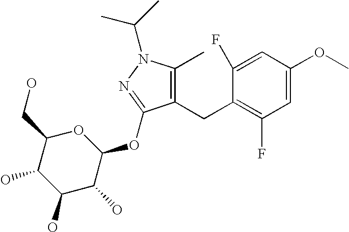

ESI-MS: m/z=459 [M+H]⁺

### (4) 4-(3,5-Difluoro-4-methoxy-benzyl)-1-isopropyl-5-methyl-3-β-D-glucopyranos-1-yloxy-1H-pyrazole

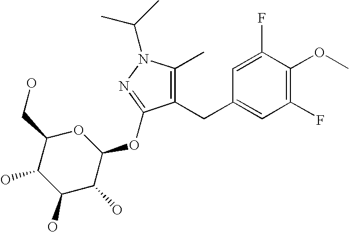

ESI-MS: m/z=459 [M+H]⁺

### (5) 1-Cyclobutyl-4-(3-fluoro-4-methyl-benzyl)-5-methyl-3-β-D-glucopyranos-1-yloxy-1H-pyrazole

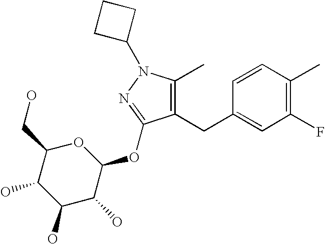

ESI-MS: m/z=437 [M+H]⁺

### (6) 1-Cyclopropylmethyl-4-(3-fluoro-4-methyl-benzyl)-5-methyl-3-β-D-glucopyranos-1-yloxy-1H-pyrazole

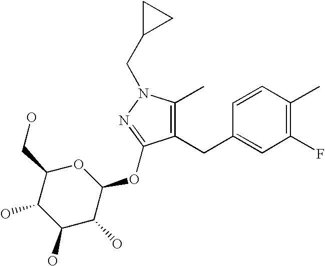

ESI-MS: m/z=437 [M+H]⁺

### (7) 1-Cyclobutyl-4-(2-fluoro-4-methoxy-benzyl)-5-methyl-3-β-D-glucopyranos-1-yloxy-1H-pyrazole

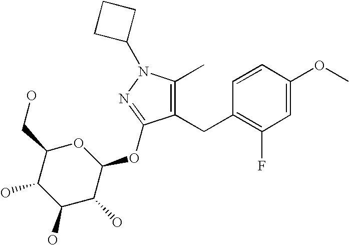

ESI-MS: m/z=453 [M+H]⁺

### (8) 4-(3-Chloro-4-methoxy-benzyl)-1-isopropyl-5-methyl-3-β-D-glucopyranos-1-yloxy-1H-pyrazole

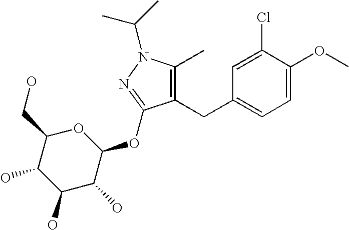

ESI-MS: m/z=457/459 [M+H]⁺ (chlorine)

### (9) 4-(2-Chloro-4-methoxy-benzyl)-1-isopropyl-5-methyl-3-β-D-glucopyranos-1-yloxy-1H-pyrazole

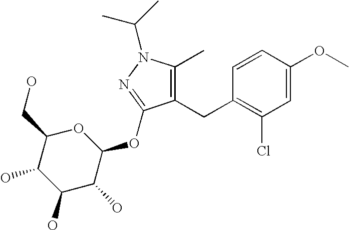

ESI-MS: m/z=457/459 [M+H]⁺ (chlorine)

### (10) 4-(4-Bromo-3-fluoro-benzyl)-1-isopropyl-5-methyl-3-β-D-glucopyranos-1-yloxy-1H-pyrazole

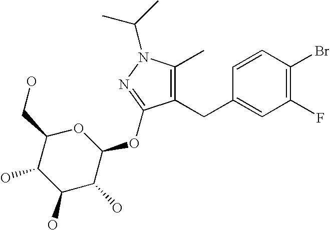

### (11) 4-(2,3-Difluoro-4-methyl-benzyl)-1-isopropyl-5-methyl-3-β-D-glucopyranos-1-yloxy-1H-pyrazole

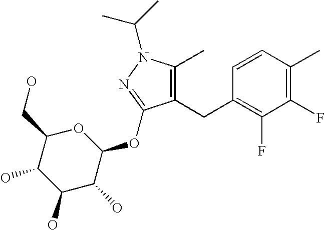

R_{f} 0.24 (silica gel, CHCl₃/MeOH 9:1)

### (12) 4-(2-Fluoro-4-methyl-benzyl)-1-isopropyl-5-methyl-3-β-D-glucopyranos-1-yloxy-1H-pyrazole

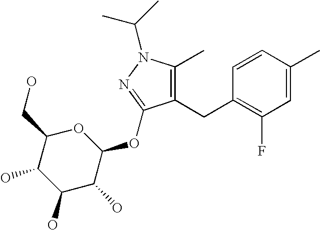

R_{f} 0.24 (silica gel, CH₂Cl₂/MeOH 9:1)

### Example 2

### (13) 4-(3-Fluoro-4-ethoxy-benzyl)-1-isopropyl-5-methyl-3-β-D-glycopyranos-1-yloxy-1H -pyrazole

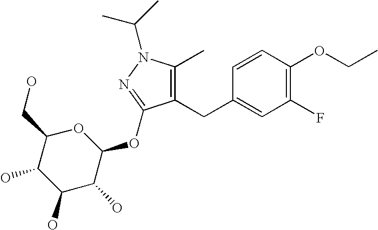

To an ice-cold solution of 4-(4-ethoxy-3-fluoro-benzyl)-1-isopropyl-5-methyl-3-(2,3,4,6-tetra-O-acetyl-β-D-glucopyranos-1-yloxy)-1H-pyrazole (0.17 g, 0.27 mmol) in MeOH (1 mL) and THF (1.5 mL) was added aqueous LiOH solution (1 M, 1.25 mL). The solution was stirred in the ice-bath for 1 h and then diluted with EtOAc and water. The organic phase was separated, washed with water and brine, and dried over Na₂SO₄. The solvent was removed, and the residue was dried in vacuo to give the product as a white foam.

Yield: 0.12 g (95%)

ESI-MS: m/z=455 [M+H]⁺

The following compounds can be obtained accordingly:

### (14) 4-(4-ethinyl-benzyl)-1-isopropyl-5-methyl-3-β-D-glucopyranos-1-yloxy-1H-pyrazole

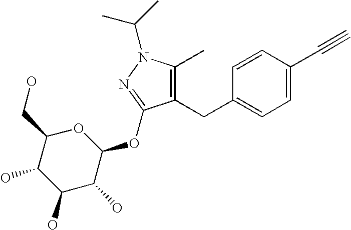

4-(4-Trimethylsilyl-ethinyl-benzyl)-1-isopropyl-5-methyl-3-(2,3,4,6-tetra-O-acetyl-β-D-glucopyranos-1-yloxy)-1H-pyrazole was subjected to the reaction conditions described above.

ESI-MS: m/z=417 [M+H]⁺

### (15) 4-(3-Fluoro-4-isopropxy-benzyl)-1-isopropyl-5-methyl-3-β-D-glucopyranos-1-yloxy-1H-pyrazole

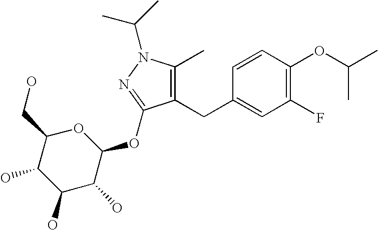

ESI-MS: m/z=469 [M+H]⁺

### (16) 4-(2-fluoro-4-methoxy-benzyl)-1-isopropyl-5-methyl-3-β-D-glucopyranos-1-yloxy-1H-pyrazole

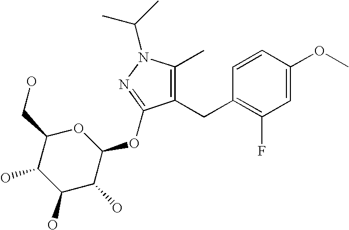

### (17) 4-(2-fluoro-4-methoxy-benzyl)-1-isopropyl-5-trifluoromethyl-3-β-D-glucopyranos-1-yloxy-1H-pyrazole

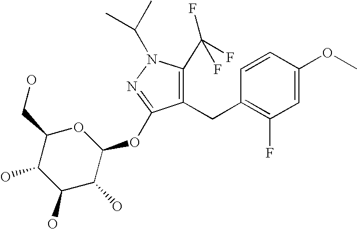

ESI-MS: m/z=495 [M+H]⁺

### (18) 4-(4-Bromo-2-fluoro-benzyl)-1-isopropyl-5-methyl-3-β-D-glucopyranos-1-yloxy-1H-pyrazole

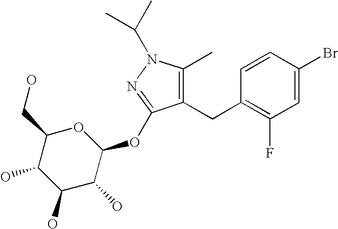

R_{f} 0.29 (silica gel, CH₂Cl₂/MeOH 9:1)

### Example 3

### (19) 4-(2-Fluoro-4-isopropoxy-benzyl)-1-isopropyl-5-methyl-3-β-D-glucopyranos-1-yloxy-1H-pyrazole

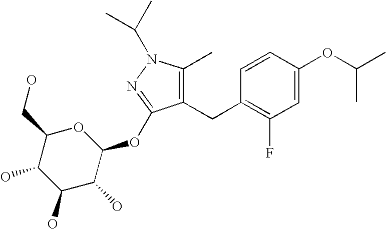

To a suspension of 4-(2-fluoro-4-hydroxy-benzyl)-1-isopropyl-5-methyl-3-β-D-glucopyranos-1-yloxy-1H-pyrazole (0.20 g, 0.47 mmol) and cesium carbonate (0.16 g, 0.50 mmol) in DMF (3.5 mL) was added isopropyl iodide (52 μL, 0.50 mmol). After stirring the mixture at ambient temperature over night, another portion of cesium carbonate (0.10 g) and isopropyl iodide (30 μL) were added. After stirring another 24 h at room temperature, the mixture was diluted with EtOAc, phosphoric acid (0.1 M), and brine. The organic phase was separated, washed with brine, and dried over Na₂SO₄. The organic solution was concentrated, and the residue was purified by silica gel chromatography (DCM/MeOH 10:1) to deliver the product as a white foam.

Yield: 0.16 g (73%)

ESI-MS: m/z=469 [M+H]⁺

The following compound can be obtained accordingly:

### (20) 4-(2-Fluoro-4-ethoxy-benzyl)-1-isopropyl-5-methyl-3-β-D-glucopyranos-1-yloxy-1H-pyrazole

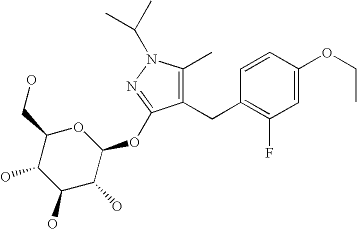

ESI-MS: m/z=455 [M+H]⁺

The compounds (21) to (29) can be obtained by methods as described in this application or in the literature.

### Example 4

### (30a) 4-(2,3-Difluoro-4-methoxy-benzyl)-1-isopropyl-5-methyl-3-(6-O-methoxycarbonyl-β-D-glucopyranos-1-yloxy)-1H-pyrazole

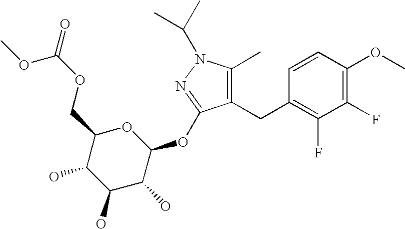

To an ice-cold solution of 4-(2,3-difluoro-4-methoxy-benzyl)-1-isopropyl-5-methyl-3-β-D-glucopyranos-1-yloxy-1H-pyrazole (0.23 g, 0.50 mmol) in 2,4,6-trimethylpyridine (0.7 mL) was added methyl chloroformate (42 μL, 0.55 mmol). The reaction solution was warmed up in the ice-bath to room temperature and stirred over night. Then the solution was diluted with Et₂O, washed with aqueous HCl (1 M) and brine, and dried over MgSO₄. The solvent was evaporated, and the residue was purified by chromatography on silica gel (DCM/MeOH 25:1→3:1) to afford the product as a white solid.

Yield: 0.15 g (56%)

ESI-MS: m/z=517 [M+H]⁺

The following compounds can be obtained accordingly:

### (31a) 4-(3-Fluoro-4-ethoxy-benzyl)-1-isopropyl-5-methyl-3-(6-O-methoxycarbonyl-β-D-glucopyranos-1-yloxy)-1H-pyrazole

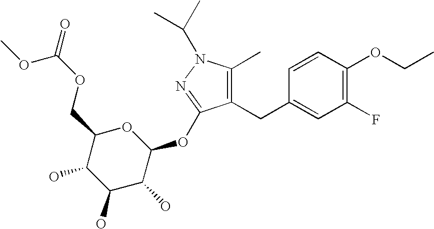

ESI-MS: m/z=513 [M+H]⁺

### (32a) 4-(3-Fluoro-4-isopropxy-benzyl)-1-isopropyl-5-methyl-3-(6-O-methoxycarbonyl-β-D-glucopyranos-1-yloxy)-1H-pyrazole

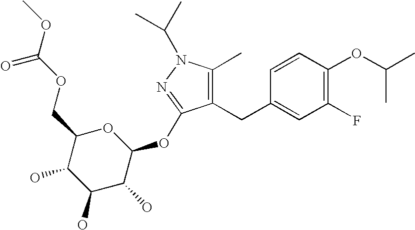

ESI-MS: m/z=527 [M+H]⁺

### (33a) 4-(2,5-Difluoro-4-methoxy-benzyl)-1-isopropyl-5-methyl-3-(6-O-methoxycarbonyl-β-D-glucopyranos-1-yloxy)-1H-pyrazole

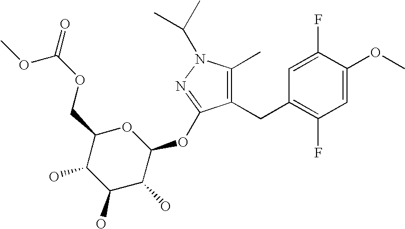

ESI-MS: m/z=517 [M+H]⁺

### (34a) 4-(2-Fluoro-4-isopropoxy-benzyl)-1-isopropyl-5-methyl-3-(6-O-methoxycarbonyl-β-D-glucopyranos-1-yloxy)-1H-pyrazole

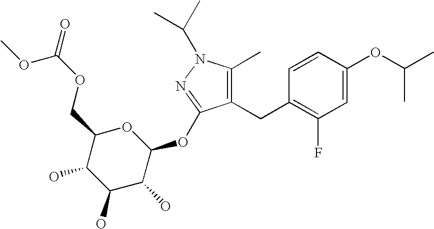

ESI-MS: m/z=527 [M+H]⁺

### (35a) 4-(2-Fluoro-4-ethoxy-benzyl)-1-isopropyl-5-methyl-3-(6-O-methoxycarbonyl-β-D-glucopyranos-1-yloxy)-1H-pyrazole

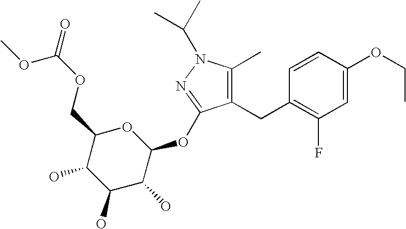

ESI-MS: m/z=513 [M+H]⁺

### (36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole

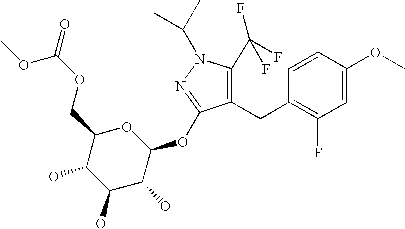

ESI-MS: m/z=553 [M+H]⁺

### (37a) 4-(2,6-Difluoro-4-methoxy-benzyl)-1-isopropyl-5-methyl-3-(6-O-methoxycarbonyl-β-D-glucopyranos-1-yloxy)-1H-pyrazole

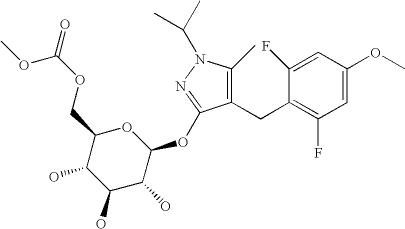

ESI-MS: m/z=517 [M+H]⁺

### (38a) 4-(3,5-Difluoro-4-methoxy-benzyl)-1-isopropyl-5-methyl-3-(6-O-methoxycarbonyl-β-D-glucopyranos-1-yloxy)-1H-pyrazole

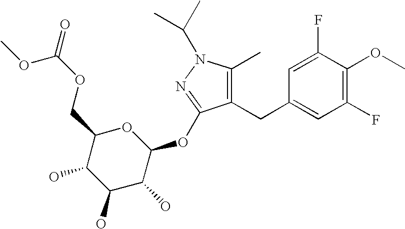

ESI-MS: m/z=517 [M+H]⁺

### (39a) 1-Cyclobutyl-4-(3-fluoro-4-methyl-benzyl)-5-methyl-3-(6-O-methoxycarbonyl-β-D-glucopyranos-1-yloxy)-1H-pyrazole

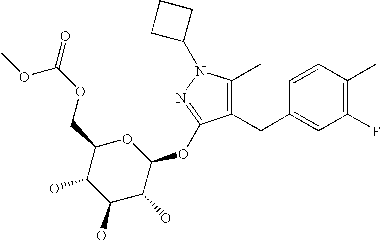

ESI-MS: m/z=495 [M+H]⁺

### (40a) 1-Cyclopropylmethyl-4-(3-fluoro-4-methyl-benzyl)-5-methyl-3-(6-methoxycarbonyl-β-D-glucopyranos-1-yloxy)-1H-pyrazole

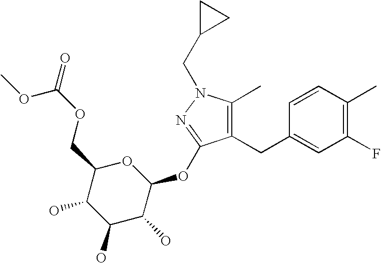

ESI-MS: m/z=495 [M+H]⁺

### (41a) 1-Cyclobutyl-4-(2-fluoro-4-methoxy-benzyl)-5-methyl-3-(6-O-methoxycarbonyl-β-D-glucopyranos-1-yloxy)-1H-pyrazole

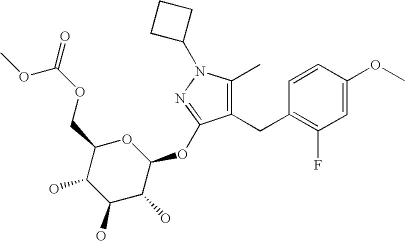

ESI-MS: m/z=511 [M+H]⁺

### (42a) 4-(4-Bromo-3-fluoro-benzyl)-1-isopropyl-5-methyl-3-(6-O-methoxycarbonyl-β-D-glucopyranos-1-yloxy)-1H-pyrazole

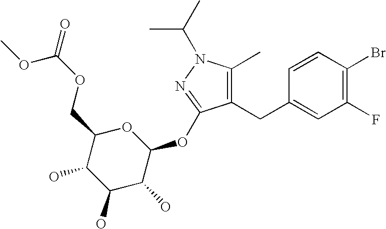

### (43a) 4-(2,3-Difluoro-4-methyl-benzyl)-1-isopropyl-5-methyl-3-(6-O-methoxycarbonyl-β-D-glucopyranos-1-yloxy)-1H-pyrazole

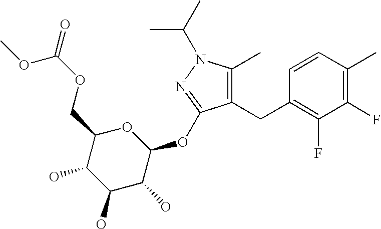

R_{f} 0.49 (silica gel, CHCl₃/MeOH 10:1)

### (44a) 4-(4-Bromo-2-fluoro-benzyl)-1-isopropyl-5-methyl-3-(6-O-methoxycarbonyl-β-D-glucopyranos-1-yloxy)-1H-pyrazole

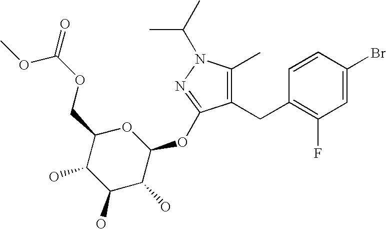

R_{f} 0.39 (silica gel, CH₂Cl₂/MeOH 19:1)

### (45a) 4-(2-Fluoro-4-methyl-benzyl)-1-isopropyl-5-methyl-3-(6-O-methoxycarbonyl-β-D-glucopyranos-1-yloxy)-1H-pyrazole

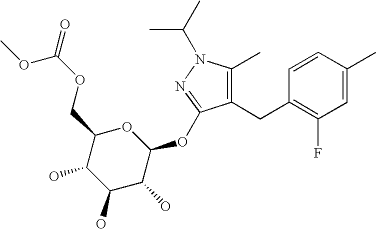

R_{f} 0.62 (silica gel, CH₂Cl₂/MeOH 9:1)

The compounds (30b), (31b), (32b), (33b), (34b), (35b), (36b), (37b), (38b), (39b), (40b), (41b), (42b), (43b), (44b) and (45b) are obtained analogously.

### Example 5

### (46) 4-(3-Fluoro-4-methyl-benzyl)-1-isopropyl-5-methyl-3-(6-O-ethoxycarbonyl-β-D-glucopyranos-1-yloxy)-1H-pyrazole

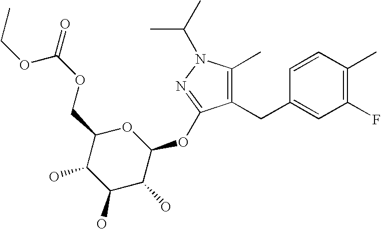

To an ice-cold solution of 4-(3-fluoro-4-methyl-benzyl)-1-isopropyl-5-methyl-3-β-D-glucopyranos-1-yloxy-1H-pyrazole (0.30 g, 0.70 mmol) in 2,4,6-trimethylpyridine (1 mL) was added methyl chloroformate (76 μL, 0.80 mmol). The reaction solution was warmed up in the ice-bath to room temperature and stirred over night. Then the solution was diluted with Et₂O, washed with aqueous HCl (1 M) and brine, and dried over MgSO₄. The solvent was evaporated and the residue was purified by chromatography on silica gel (DCM/MeOH 25:1→3:1) to afford the product as a white solid.

Yield: 0.23 g (66%)

ESI-MS: m/z=497 [M+H]⁺

The following compounds can be obtained by analogy with the procedure described above:

### (47) 4-(2-Fluoro-4-methoxy-benzyl)-1-isopropyl-5-methyl-3-(6-O-ethoxycarbonyl-β-D-glucopyranos-1-yloxy)-1H-pyrazole

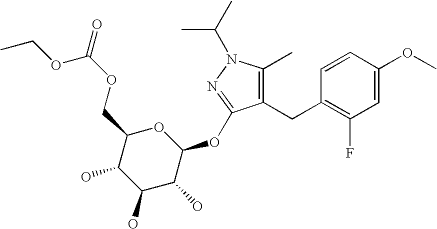

ESI-MS: m/z=513 [M+H]⁺

### (48) 4-(2-Fluoro-4-methoxy-benzyl)-1-isopropyl-5-methyl-3-(6-O-isobutyloxycarbonyl-β-D-glucopyranos-1-yloxy)-1H-pyrazole

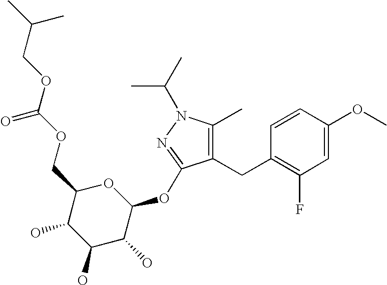

ESI-MS: m/z=541 [M+H]⁺

### (49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole

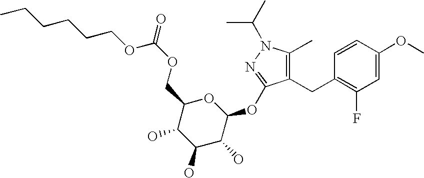

ESI-MS: m/z=569 [M+H]⁺

### (50) 4-(2-Fluoro-4-methoxy-benzyl)-1-isopropyl-5-methyl-3-(6-O-phenoxycarbonyl-β-D-glucopyranos-1-yloxy)-1H-pyrazole

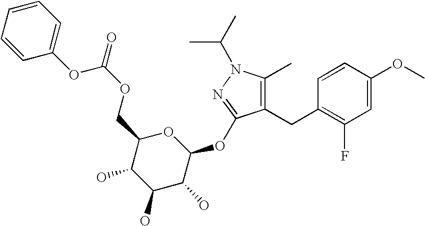

ESI-MS: m/z=561 [M+H]⁺

### (51) 4-(2-Fluoro-4-methoxy-benzyl)-1-isopropyl-5-methyl-3-(6-O-benzyloxycarbonyl-β-D-glucopyranos-1-yloxy)-1H-pyrazole

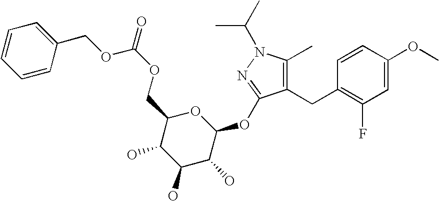

ESI-MS: m/z=575 [M+H]⁺

### (52) 4-(2-Fluoro-4-methoxy-benzyl)-1-isopropyl-5-methyl-3-(6-O-acetyl-β-D-glucopyranos-1-yloxy)-1H-pyrazole

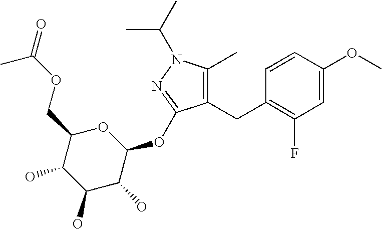

ESI-MS: m/z=483 [M+H]⁺

### (53) 4-(2-Fluoro-4-methoxy-benzyl)-1-isopropyl-5-methyl-3-(6-O-propylcarbonyl-β-D-glucopyranos-1-yloxy)-1H-pyrazole

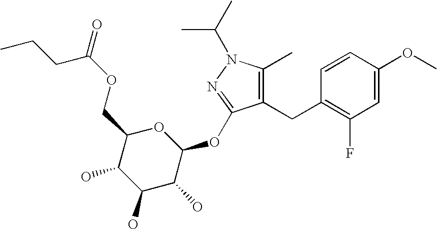

ESI-MS: m/z=511 [M+H]⁺

### (54) 4-(2-Fluoro-4-methoxy-benzyl)-1-isopropyl-5-methyl-3-(6-O-isopropylcarbonyl-β-D-glucopyranos-1-yloxy)-1H-pyrazole

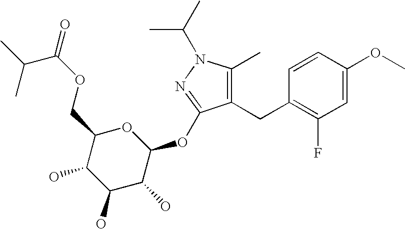

ESI-MS: m/z=511 [M+H]⁺

### (55) 4-(2-Fluoro-4-methoxy-benzyl)-1-isopropyl-5-methyl-3-(6-O-benzylcarbonyl-β-D-glucopyranos-1-yloxy)-1H-pyrazole

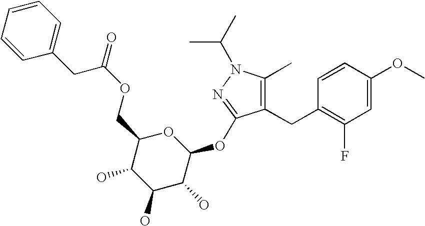

ESI-MS: m/z=559 [M+H]⁺

The compounds (56) to (63) can be obtained by analogy with the procedure described above.

### Examples of Formulations

The following examples of formulations, which may be obtained analogously to methods known in the art, serve to illustrate the present invention more fully without restricting it to the contents of these examples. The term “active substance” denotes one or more compounds according to the invention, i.e. denotes a pyrazole-O-glucoside derivative according to this invention or a second therapeutic agent according to this invention or a combination of said pyrazole-O-glucoside derivative with said second therapeutic agent, for example selected from the combinations 1a to 7h as listed in Table 1. Additional suitable formulations for the second therapeutic agent may be those formulations which are available on the market or formulations described in the literature, for example as disclosed in current issues of “Rote Liste®” (Editio Cantor Verlag Aulendorf, Germany) or of “Physician's Desk Reference”.

### Example 1

### Dry Ampoule Containing 75 mg of Active Substance Per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### Example 2

### Dry Ampoule Containing 35 mg of Active Substance Per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use, the product is dissolved in water for injections.

### Example 3

### Tablet Containing 50 mg of Active Substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### Example 4

### Tablet Containing 350 mg of Active Substance

Preparation:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 5

### Capsules Containing 50 mg of Active Substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing. This powder mixture is packed into size 3 hard gelatin capsules in a capsule filling machine.

### Example 6

### Capsules Containing 350 mg of Active Substance

Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

Preparation:

(1) is triturated with (3). This trituration is added to the mixture of (2) and (4) with vigorous mixing. This powder mixture is packed into size 0 hard gelatin capsules in a capsule filling machine.

### Example 7

### Suppositories Containing 100 mg of Active Substance

Composition:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

1. A pharmaceutical composition comprising a pyrazole-O-glucoside derivative selected from the group of compounds (1) to (29) consisting of
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(16) 4-(2-fluoro-4-methoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
(21) 4-(4-ethyl-benzyl)-1-isopropyl-5-trifluoromethyl-3-β-D-glucopyranos-1-yloxy-1H-pyrazole;
(22) 4-(4-bromo-benzyl)-1-isopropyl-5-trifluoromethyl-3-β-D-glucopyranos-1-yloxy-1H-pyrazole;
(23) 4-(4-ethyl-benzyl)-1-cyclobutyl-5-trifluoromethyl-3-β-D-glucopyranos-1-yloxy-1H-pyrazole;
(24) 4-(4-ethyl-benzyl)-1-(2-fluoro-1-fluoromethyl-ethyl)-5-trifluoromethyl-3-β-D-glucopyranos-1-yloxy-1H-pyrazole;
(25) 4-(3-fluoro-4-methoxy-benzyl)-1-isopropyl-5-trifluoromethyl-3-β-D-glucopyranos-1-yloxy-1H-pyrazole;
(26) 4-(3-fluoro-4-methyl-benzyl)-1-isopropyl-5-methyl-3-β-D-glucopyranos-1-yloxy-1H-pyrazole;
(27) 4-(2,3-difluoro-4-isopropoxy-benzyl)-1-isopropyl-5-methyl-3-β-D-glucopyranos-1-yloxy-1H-pyrazole;
(28) 4-(3-fluoro-4-methoxy-benzyl)-1-isopropyl-5-methyl-3-β-D-glucopyranos-1-yloxy-1H-pyrazole;
(29) 4-(4-ethyl-benzyl)-1-isopropyl-5-methyl-3-β-D-glucopyranos-1-yloxy-1H-pyrazole;
or a prodrug thereof wherein one or more hydroxyl groups of the β-D-glucopyranosyl group are acylated with groups selected from (C₁₋₃-alkyl)carbonyl, (C₁₋₆-alkyl)-oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof;
in combination with at least one second therapeutic agent which is suitable in the treatment or prevention of one or more conditions selected from type 1 diabetes mellitus, type 2 diabetes mellitus, impaired glucose tolerance (IGT), impaired fasting blood glucose (IFG), and hyperglycemia.

2. The pharmaceutical composition according to claim 1 characterized in that the at least one second therapeutic agent is selected from the groups consisting of
a) biguanides,
b) sulfonylureas,
c) metiglinides,
d) thiazolidindiones,
e) alpha-glucosidase inhibitors,
f) insulins and insulin analogues,
g) GLP1 and GLP1 analogues,
h) PPAR gamma modulators,
i) PPAR gamma/alpha modulators,
j) glucose-dependent insulinotropic polypeptide agonists,
k) beta-3 agonists, and
l) dipeptidyl peptidase IV inhibitors.

3. The pharmaceutical composition according to claim 2 characterized in that the at least one second therapeutic agent is selected from the groups consisting of:
a) metformin, phenformin, buformin;
b) glibenclamide, tolbutamide, glimepiride, glipizid, gliquidon, glibornurid, glyburide, gliclazid, nateglinide, repaglinide;
c) nateglinide, repaglinide;
d) pioglitazone, rosiglitazone, troglitazone, ciglitazone;
e) miglitol, acarbose, voglibose;
f) human insulin, insulin lispro, insulin glusilin, recombinant insulins, insulin aspart, NPH insulin, insulin detemir, insulin zinc suspension and insulin glargin;
g) exendin-4;
h) metaglidasen;
i) tesaglitazar, muraglitazar, KRP297;
j) pramlintide, amlyin;
k) ritobegron, YM 178, solabegron, talibegron, N-5984, GRC-1087, rafabegron, FMP825;
l) sitagliptin, vildagliptin, saxagliptin and alogliptin.

4. The pharmaceutical composition according to claim 1 characterized in that the at least one second therapeutic agent is metformin or insulin.

5. The pharmaceutical composition according to claim 1 characterized in that the at least one second therapeutic agent is pioglitazone or rosiglitazone.

6. The pharmaceutical composition according to claim 1 characterized in that the at least one second therapeutic agent is glimepiride, miglitol, voglibose or acarbose.

7. The pharmaceutical composition according to claim 1 characterized in that the composition is suitable for combined or simultaneous or sequential use of the pyrazole-O-glucoside derivative and the at least one second therapeutic agent.

8. The pharmaceutical composition according to claim 1 characterized in that the pyrazole-O-glucoside derivative and the at least one second therapeutic agent are present in a single dosage form.

9. The pharmaceutical composition according to claim 1 characterized in that the pyrazole-O-glucoside derivative and the at least one second therapeutic agent are present each in a separate dosage form.

10. Method for preventing, slowing the progression of, delaying or treating a metabolic disorder selected from the group consisting of type 1 diabetes mellitus, type 2 diabetes mellitus, impaired glucose tolerance, impaired fasting blood glucose, hyperglycemia, postprandial hyperglycemia, overweight, obesity and metabolic syndrome comprising administering to a patient in need thereof a pharmaceutical composition according to claim 1.

11. Method for improving glycemic control and/or for reducing of fasting plasma glucose, of postprandial plasma glucose and/or of glycosylated hemoglobin IIbA1c comprising administering to a patient in need thereof a pharmaceutical composition according to claim 1.

12. Method for preventing, slowing, delaying or reversing progression from impaired glucose tolerance, impaired fasting blood glucose, insulin resistance and/or from metabolic syndrome to type 2 diabetes mellitus comprising administering to a patient in need thereof a pharmaceutical composition according to claim 1.

13. Method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, comprising administering to a patient in need thereof a pharmaceutical composition according to claim 1.

14. Method for reducing the weight or preventing an increase of the weight or facilitating a reduction of the weight comprising administering to a patient in need thereof a pharmaceutical composition according to claim 1.

15. Method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion comprising administering to a patient in need thereof a pharmaceutical composition according to claim 1.

16. Method for preventing, slowing, delaying or treating diseases or conditions attributed to an abnormal accumulation of liver fat comprising administering to a patient in need thereof a pharmaceutical composition according to claim 1.

17. Method for maintaining and/or improving the insulin sensitivity and/or for treating or preventing hyperinsulinemia and/or insulin resistance comprising administering to a patient in need thereof a pharmaceutical composition according to claim 1.

18. (canceled)

19. (canceled)

20. (canceled)

21. Method according to claim 1, wherein the patient is an individual diagnosed of one or more of the conditions selected from the group consisting of overweight, obesity, visceral obesity and abdominal obesity.

22. Method according to claim 1, wherein the patient is an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%.

23. Method according to claim 1, wherein the patient is an individual wherein one, two, three or more of the following conditions are present:
(a) obesity, visceral obesity and/or abdominal obesity,
(b) triglyceride blood level≧150 mg/dL,
(c) HDL-cholesterol blood level<40 mg/dL in female patients and <50 mg/dL in male patients,
(d) a systolic blood pressure≧130 mm Hg and a diastolic blood pressure≧85 mm Hg,
(e) a fasting blood glucose level≧110 mg/dL.

24. Method according to claim 1, wherein the patient is an individual for whom the monotherapy with metformin is contraindicated and/or who has an intolerance against metformin at therapeutic doses.

25. Method according to claim 1, wherein the patient is an individual with insufficient glycemic control despite treatment with one or more antidiabetic drugs selected from the groups a) to l)
a) biguanides,
b) sulfonylureas,
c) metiglinides,
d) thiazolidindiones,
e) alpha-glucosidase inhibitors,
f) insulins and insulin analogues,
g) GLP1 and GLP1 analogues,
h) PPAR gamma modulators,
i) PPAR gamma/alpha modulators,
j) glucose-dependent insulinotropic polypeptide agonists,
k) beta-3 agonists, and
l) dipeptidyl peptidase IV inhibitors
or
a) metformin, phenformin, buformin;
b) glibenclamide, tolbutamide, glimepiride, glipizid, gliquidon, glibornurid, glyburide, gliclazid, nateglinide, repaglinide;
c) nateglinide, repaglinide;
d) pioglitazone, rosiglitazone, troglitazone, ciglitazone;
e) miglitol, acarbose, voglibose;
f) human insulin, insulin lispro, insulin glusilin, recombinant insulins, insulin-aspart, NPH insulin, insulin detemir, insulin zinc suspension and insulin glargin;
g) exendin-4;
h) metaglidasen;
i) tesaglitazar, muraglitazar, KRP297;
j) pramlintide, amlyin;
k) ritobegron, YM 178, solabegron, talibegron, N-5984, GRC-1087, rafabegron, FMP825;
l) sitagliptin, vildagliptin, saxagliptin and alogliptin.

26. Method according to claim 1, wherein the at least one second therapeutic agent is metformin or insulin.

27. Method according to claim 1, wherein the at least one second therapeutic agent is pioglitazone or rosiglitazone.

28. Method according to claim 1, wherein the at least one second therapeutic agent is miglitol, glimepiride, voglibose or acarbose.
